(19) United States
(12) Patent Application Publication
(10) Pub. No.: US 2019/0139642 A1
(43) Pub. Date: May 9, 2019

(54) SYSTEM AND METHODS FOR MEDICAL IMAGE ANALYSIS AND REPORTING

(71) Applicant: Ascend Hit LLC, Lisle, IL (US)

(72) Inventors: James ROBERGE, Lisle, IL (US); Jeffrey SOBLE, Lisle, IL (US); James WOLFER, Berrien Springs, MI (US)

(21) Appl. No.: 16/096,129

(22) PCT Filed: Apr. 26, 2017
(86) PCT No.: PCT/US17/29689

§ 371 Date: Oct. 24, 2018

Related U.S. Application Data
(60) Provisional application No. 62/327,743, filed on Apr. 26, 2016.
(60) Provisional application No. 62/341,698, filed on May 26, 2016.

(51) Int. Cl. G16H 30/40; G16H 15/00; G06F 3/01; G06F 3/16; G16H 30/20; G06N 3/08

(52) U.S. Cl. CPC G16H 30/40; G16H 15/00; G06N 3/08; G06F 3/16; G16H 30/20; G06F 3/013

(57) ABSTRACT

The present invention relates generally to a system and methods for medical image analysis and reporting. Specifically, certain preferred embodiments relate to a system that is configurable to receive a variety of inputs such that a user may choose the images, information, data, or other content reviewed by the user, the information that will result from that review and be inputted into the system, and the type of report that may be generated. In certain embodiments, the system facilitates matching the inputs to terms of a set of structured data elements to produce one or more templates that a user may select. An identifier including at least one term from the selected template may be assigned to the image. The system may then access an identified image database and process images with the identifier to produce a machine learning model, which can then be used to train a machine learning algorithm.

### CROSS-REFERENCE TO RELATED APPLICATIONS

This application claims the benefit of U.S. Provisional Patent Application No. 62/327,743 filed Apr. 26, 2016 and U.S. Provisional Patent Application No. 62/341,698 filed May 26, 2016, each of which is incorporated by reference.

### FIELD OF INVENTION

The present invention relates generally to medical image analysis and reporting. In particular, certain preferred embodiments of the present invention relate to a system that is configurable to receive a variety of inputs such that a user may choose the images, information, data, or other content reviewed by the user, the information that will result from that review and be inputted into the system, and the type of report that may be generated. Examples of inputs that the system can receive include mouse clicks, typed text, touch, gestures, utterances, gaze data, image data and further includes imaging modality, image projections, anatomy, functional parameters, geometric parameters, and pathologies. Examples of that which the system may develop include one or more phrases, sentences, paragraphs, and further include visual representations that present, explain, or put into context the information received by the system. Advantageously, the inputs that may be developed through the use of the system may be used to train Deep Learning models that may automatically identify structured data elements such as, but not limited to regions or objects within images.

### BACKGROUND OF THE INVENTION

In many industries, professionals are engaged to consider and produce a report of their observations or findings regarding certain materials, information, data, and other content. Such other content may include static or dynamic images, text, and/or transcripts of verbal communications.

In the medical industry, professionals may consider a range of images (e.g., X-ray, CT, MRI, and ultrasound images) such as those that provide a detailed view of a patient's anatomy. The review of medical images by trained cardiologists or radiologists, for example, is an essential, routine step in diagnosing pathology, guiding treatment, and evaluating treatment outcomes.

During the course or at the completion of the review and examination of the medical images, radiologists can use a variety of tools to record their observations and findings including handwriting, typing, dictation, and speech recognition systems.

However, the use of conventional tools to record information is often a time consuming process. Medical professionals typically need to review large volumes of images and information on a daily basis. Recording options that may take less time would provide a variety of advantages to the professional.

Also, many traditional tools that are available to professionals to record information do not facilitate such recording without errors. Specifically, recording tools such as dictation, speech recognition, and structured reporting produce reports that often include spelling and grammatical errors.

Additionally, tools that professionals may use to record observations and findings while viewing images and other content may require the professional to constantly shift their focus while recording information to ensure that the information is being accurately reported. This may also limit the speed and accuracy with which reports can be created.

Therefore, there is a need for a system that facilitates the recording of observations and findings and the production of a report that is not time consuming and limits or eliminates errors and that is configurable such that the user can select the content that is being observed, the input or inputs that are produced from such observations, and the report that results from the review. The present invention satisfies this demand.

### SUMMARY

The present invention relates to a system and methods that facilitate that development of one or more inputs as a result of image analysis and the recording of observations and/or findings from a medical image. Specifically, certain preferred embodiments of the present invention relate to a system that is configurable to choose the images, information, data, or other content reviewed by the user, the information that will result from that review and be inputted into the system, and the type of report that may be generated.

In certain embodiments, the system can display a selected image and analyze at least one input to identify a region of interest (“ROI”) and accurately match words of a recorded utterance to one or more structured data elements from a set of structured data elements in order to produce an output corresponding to the inputs. This process is referred to, for purposes of this application, as mapping one or more inputs to a structured data set.

Certain preferred embodiments of the invention relate to a system and methods by which at least one input is analyzed, identified, and matched to at least one structured data element from a set of structured data elements. An input refers to the data, image, content, or other information received by a computing device or a component of the computing device from another device or from a piece of software—either automatically or manually. Examples of inputs that the system can receive include mouse clicks, typed text, touch, gestures, utterances, gaze data, image data and further includes imaging modality, image projections, anatomy, functional parameters, geometric parameters, and pathologies.

For purposes of this application, the term “ROI” refers to a selected subset of data from an image data set that identifies the borders of an object under consideration. For purposes of this application, the term “image” may include one or more words, phrases, sentences, numbers, symbols, icons, pictures, graphics, and the like.

Certain embodiments of the present invention may facilitate image processing such that boundaries, contours, shapes, or configurations of an anatomical feature or pathology are automatically detected and distinguished. In one example, image information may be extracted from the ROI through the use of an image processing algorithm.

Certain embodiments of the present invention facilitate the use of a structured reference frame to identify the position and geometric structure of a feature—such as, an anatomical feature or pathology—in the one or more images that were produced using different imaging modalities (e.g., x-ray, ultrasound, MRI). Information from a number of regions of interest may then be provided in a reference frame that can be associated with a user's point of gaze. In some embodiments, adding spatial locations to a ROI identifies an image processing region. The system can then identify an ordered set of regions of interest within the image.

In certain preferred embodiments, the system may use gaze data to determine what a user is viewing on a display. Collected and analyzed gaze data may be used to determine one or more coordinates on a image presented on a display to a user. The system can then identify a ROI by the physical coordinate—one point or an average of many points on the image that correspond to the user's point of gaze. For example, the system can identify the ROI based on the detected coordinates of a user's gaze and a distance of the coordinates from one or more boundaries of an object within an image. Alternatively, the system may access a ROI database and match one or more coordinates of the user's gaze to a ROI database entry to identify the ROI. In some embodiments, the system may use the physical coordinates to identify multiple regions of interest.

In certain embodiment, a “gaze profile” which includes gaze positions, velocities of eye movements, and dwell times of a user may help identify or categorize ROIs within one or more images sets, prior reports, or the electronic medical record. The system may then use the gaze profile for administrative purposes, or store the gaze profile for possible access by other physicians.

Using the gaze profile, physicians can convey a related set of information to health care providers or identify a data set that the original reviewing physician found interesting. For example, a professional may associate information with ROIs as guideposts for junior physicians or students who may be less experienced in reading an image. In certain embodiments, the original physician may manually select the most important ROI from the set of auto-generated ROIs. Alternatively, the spoken utterances made while an ROI is being examined or the structured data associated with the ROI can be displayed when the ROI is detected in the future.

In certain embodiments, the system may further analyze the recorded gaze data to detect patterns of visual fixations and saccades in the gaze data to identify the region of interest.

Certain patterns may also be indicative of indecision on the part of the reading physician, suggesting a less confident diagnosis. The system may collect and analyze gaze profiles with the validated diagnosis data to provide the basis for feedback and/or a confidence metric.

In another embodiment of the current invention, the tracking and recording of gaze data also provides additional useful context about non-image data that the user is examining—including, but not limited to, information in prior reports and on-screen displays of data about the patient (e.g., measurements, data from the electronic health record or other sources), as well as data entry forms.

In certain embodiments, the system may automatically associate the ROI with information—such as, clinical information and/or a structured data element. For example, one or more ROIs may be associated with an identifier—such as a digital object identifier—that is linked to metadata corresponding to clinical information. In some embodiments, the identifier may be a unique identifier linked to a certain patient and/or certain patient information. The system may receive a user's gaze data, analyze the gaze data, identify a ROI, detect an identifier and, in response, locate and provide the user with relevant information—such as a patient's clinical history. For example, the system may identify a ROI by detecting a certain identifier (number, icon, name, etc.) displayed in association with the image, and, in response, output information about a patient, such as clinical information located in the database. In another example, the system may use the radiologist's gaze data to identify a certain anatomical feature and detect an identifier that is linked to metadata corresponding to information about the anatomical feature and/or patient information regarding that anatomical feature.

In some embodiments, the system may associate an identifier with an anatomical term or context, which may be used by speech recognition software. For example, given a context, speech recognition software may accurately and reliably record the meaning of an utterance.

In another embodiment of the current invention, a gesture is used either with or without gaze data to detect an identifier or identify a ROI that may provide additional context for matching an input to a structured data element. This can be accomplished through any gesture recognition interface and might include holographic, virtual reality, and printed renderings of the data.

The present invention is also configurable to receive and analyze an input—such as, image data, ROI data, utterances, or combinations of each—and match the input to one or more terms of a structured data set. A template is then selected based on how well the inputs match the structure, semantics, context, and content of the terms within the structured data set.

For purposes of this application, an “utterance” refers to a verbalization of a word or a sequence of words spoken by a user that can be converted to text by speech recognition software. The utterance can be entered or inputted into a system through an audio pickup such as a microphone. The system also permits entry of observations through standard input devices, for example, a mouse, keyboard, touch-screen, or any other user interface.

According to the invention, a “structured data set” refers to a hierarchy of structured data elements and may further include a sub-hierarchy of structured data elements. A structured data element is selected from a set of structured data elements that may match one or more inputs. The system may compare the one or more inputs to each structured data element and, using a scoring metric, as described below, the structured data element with the highest score is selected—either automatically by the system or in response to a user selection. The score of each structured data element in the set of structured data elements is determined using a matching algorithm as further described in detail below. For purposes of this application, a structured data element is used interchangeably with the word “term” and includes one or more words, phrases, sentences and may include numbers, symbols, icons, pictures, and graphics.

Once the one or more inputs are matched to a structured data element within a set of structured data elements, a template can be generated such as a narrative template or a report template. In certain embodiments, a narrative or report can be quickly generated without the need of further interaction by a user (e.g., a user selection). A “narrative template” represents narrative text. Narrative text is one or more phrases, sentences, paragraphs, or graphical representations and may include fields denoting at least one shortcut or placeholder such as a blank slot or pick-list. For example, a narrative template may be a “fill-in-the-blank” sentence in which the template corresponds to an organ and the blank slots or fields are filled with properties that describe that organ. A narrative is a representation such as a verbal or visual representation of the narrative template. A “report template” is a template that represents the structure of a report such as layout and format and may further include a narrative template or set of narrative templates associated with that report. Reports are typically a visual representation that can be generated to present, explain, or put into context various types of information including data, results of tests, information regarding procedures, and the status of the subject.

Each template within the set of templates includes a structure that may be represented by nodes. More specifically, the set of structured data elements of the present invention includes a widely-used data structure that emulates a hierarchical tree structure with a set of linked nodes. A root node is the topmost node in the hierarchical tree structure. According to certain preferred embodiments of the invention, root nodes typically signify one or more templates and nodes below the root nodes signify either fields within the one or more templates, a set of structured data elements that can be used to populate one or more fields of one or more templates, or the root nodes of sub-hierarchies of one or more templates.

A node may also represent a separate data structure (which could be a hierarchical tree structure of its own). Each node in a tree has zero or more child nodes, which are below it in the hierarchical tree structure. A parent node is a node that has a child node whereas a leaf node—or terminal node—is a node that does not have any children.

Each node in the set of structured data elements is typically bound to one or more data elements, which may be coded data elements—that is, data elements which are mapped to a database schema or coding standard. The data element is either a term or a sub-hierarchy root, wherein the sub-hierarchy root further includes nodes representing data elements. According to the invention, sub-hierarchy root nodes typically signify one or more sub-templates, which is a template within a template. It is also contemplated that the sub-hierarchy may further include data elements representing additional roots of additional sub-hierarchies, etc. The template defines how data elements are related and can also describe how the associated narrative or report is generated from data elements populated within a template.

Certain preferred embodiments of the invention select a template based on the extent to which the one or more inputs match the structure, semantics, content, and context of the template. In addition, the structure, semantics, content, and context of the template may be used to guide the process of selecting individual nodes from the set of structured data elements in order to select and populate the template.

In certain embodiments, an image or ROI may include an identifier that is automatically linked with one or more templates. To illustrate the use of one preferred embodiment of the invention, a system can analyze the gaze data of a radiologist looking at the left coronary artery displayed on a medical image and, using the gaze data, identify the ROI as the left anterior descending (“LAD”) of the left coronary artery, detect an identifier associated with the LAD, and output a template linked to the ROI.

In certain preferred embodiments, the matching terms—including the consideration of structure, semantics, context, and content—of the template correlate to data elements that are used to populate the template, which are then recorded for use such as to communicate the narrative template or report template. As described above, the communicated narrative template is simply termed “narrative” and the communicated report template is termed “report”. In certain preferred embodiments, the system communicates both the narrative templates and report templates.

Certain preferred embodiments of the present invention utilize a matching algorithm with a scoring metric to determine exact, incomplete, and ambiguous matches of terms in the set of structured data elements including any terms within any sub-hierarchies. The matching algorithm may also account for instances where only portions of an input match any terms of a hierarchy and any sub-hierarchies.

In certain embodiments, the system may identify incomplete or ambiguous matches between the input data and the output template. For example, not all the text derived from a speech utterance can be matched to the structured data and the associate lexical output, the data derived from the associated image or ROI does not match, or the data is contradictory to matching structured data, or there are ambiguous matches—a low level of confidence. The system may resolve such inconsistencies by one of several mechanisms.

One solution includes provisionally supplementing the structured data output with unstructured text or highlighting areas of concern within the formatted report. In another option, the system may present the user interface elements on a display in order to highlight inconsistencies and allow them to be resolved by the user.

As mentioned above, eye tracking and associated image data may improve the accuracy of speech recognition software—creating a higher confidence level in the selected template. In certain embodiments, some or all of the speech to text output may remain free text unassociated with structured data for the purposes of report narrative generation. Although the narrative of the report may be free text, rather than text generated by matching to structured data that produces a report output, the free text can still be used to infer structured data for purposes other than the report narrative—for instance, analytics or as inputs to clinical decision support rules.

Certain preferred embodiments match the one or more inputs against each structured data element of the set of structured data elements and deconstruct it into a set of sub-problems in which the one or more inputs are matched against each of the structured data element of the set of structured data elements and sub-hierarchies. In certain embodiments, features of the data—such as image data and/or ROI data—may serve as an input into the system to narrow the possible terms of the set of structured data elements and sub-hierarchies. The deconstruction process continues recursively downward through the set of structured data elements until a leaf node is reached, where each leaf node requires scoring the relative match between the terms of the leaf node and the one or more inputs into the system.

The resulting score of the match between terms of each set of structured data elements and the one or more inputs may be propagated upward in the set of structured data elements and used to compute scores for successively higher terms of non-leaf nodes, where each hierarchy and sub-hierarchy is scored after being populated in the template. In embodiments that include a set of structured data elements with sub-hierarchies, the highest-scoring term or set of terms of each template sub-hierarchy is selected and passed upwards. This upward propagation and refinement of matching terms continues until the root node is encountered, at which point the populated template is scored against all the inputs in a total score for the template including all sub-hierarchy terms. The present invention applies this hierarchical matching process to a set of structured data elements, including all of their sub-hierarchies, and selects the highest-scoring populated template.

If two or more templates achieve the same score, or there is an ambiguity in the matching process, additional information from the image and/or ROI may serve as an input into the matching algorithm to narrow the template choices for matching. For example, certain features including imaging modality, image projection, anatomy, functional parameters, geometric parameters, and pathology may serve as inputs into the matching algorithm. The system may then display certain templates for the user to review, select, and/or edit.

As mentioned above, in preferred embodiments of the present invention, the system may automatically select a template from the set of structured data elements based on scoring, with the highest scoring template selected. A score is determined as the template corresponding to each set of structured data elements is filled-in, with the terms of the hierarchy corresponding to the one or more inputs. As an example, given the utterance “small mass that looks round”, the user's gaze data identifying the ROI as the LAD, and the following pathology template:
[PATHOLOGY]=[SIZE] [SHAPE] mass in the [LOCATION]
   the system decomposes the problem of matching the inputs—the utterance and the ROI—to the set of structured data elements into three problems, or sub-problems. Each structured data element in each set of structured data elements or sub-hierarchy is represented by a structured data element vector. A structured data element vector also represents the ROI. A structured data element vector similarly represents each word in the utterance. The degree to which the utterance, the ROI, or a combination of both match a set of structured data elements or sub-hierarchy is obtained based on the intersection and dot product of the ROI term vector and the template term vector, the utterance term vector and the template term vector, and/or a combination of both.

In the example above, the set of structured data elements for the [SHAPE] field includes three terms: small, medium, large, the set of structured data elements for the [SHAPE] field includes three terms: oval, round, tubular, and the set of structured data elements of the [LOCATION] field includes two terms: left anterior descending, circumflex. The utterance is first matched against the set of structured data elements for the [SIZE] field, which yields the match “small” with a term vector dot product score of approximately 1.0, which signifies a perfect match. Second, the utterance is matched against the set of structured data elements for the [SHAPE] field, which yields the match “round” with a term vector dot product score of approximately 1.0, which signifies a perfect match. The ROI data is then matched against the set of structured data elements for the [LOCATION] field, which yields the match “left anterior descending” with the term vector dot product of approximately 1.0, which signifies a perfect match. Lastly, the utterance and the ROI is matched against the entire template after filling-in or populating the template with the data elements obtained for the [SIZE], [SHAPE], and [LOCATION] sub-problems above, where said data elements may be coded data elements. Population of the template is accomplished by recursively traversing the set of structured data elements and selecting the highest scored matched result for each hierarchy and sub-hierarchy. The populated template has the following associated data elements:
LOCATION=left anterior descending
SIZE=small
SHAPE=round
PATHOLOGY=mass

The utterance and the ROI, in the example above, is matched against the populated template “small round mass in the left anterior descending”, which yields a term vector dot product or total score of approximately 1.0, signifying a perfect match to the template. The term vector dot product is computed using the term vector corresponding to the set of terms in the populated set of structured data elements and the term vector corresponding to the set of terms in the utterance and the ROI. In the example above, the template is an exact match.

In instances where the populated template is not an exact match, the utterance and ROI may be matched against additional templates from the set of structured data elements. The system may then review the results, populate the template with the highest score, and prompt the user for confirmation. Alternatively, the system can further analyze the image data to identify certain features of the image that can serve as inputs into the matching algorithm. For example, the system can determine that the image data narrows the templates to terms associated with a certain area of the body—such as the heart. Then the system may display the possible matching templates such that a user may select one or more templates. Upon selecting the populated template, the resulting data elements (which may be coded data elements) are recorded into memory such that the narrative “small round mass in the left anterior descending” is available for to the user through a number of outputs—such as audibly through a speaker or visually on a display.

Certain embodiments of the present invention are also configurable to assign an identifier—such as a term from the selected template—to the image or a ROI and link that identifier to the recorded template stored in memory. Using that identifier, the system may train a machine learning algorithm to improve medical analysis and reporting. For example, the system can use one or more data sets including medical images with a certain identifier to find and extract patterns automatically for use in a classification or regression process.

While a number of systems utilize eye tracking technology, existing systems are not combined with other inputs for medical image analysis and reporting. Specifically, the context within which an utterance is spoken—such as a particular ROI—is not used as an input into the system.

One of the main advantages of using a variety of inputs is that reports—such as those in the medical industry—can be created accurately and efficiently. For example, eye tracking technology and speech recognition creates a complete “eyes on the image” paradigm for a medical professional that facilitates the recording of observations and findings while the professional remains focused on the image.

It is contemplated that the resulting system may use information from an image or a certain ROI to train, augment, and/or validate machine learning algorithms for automatic detection and classification of pathologic conditions. One such machine learning algorithm, Deep Learning (DL), involves training a computer to recognize often complex and abstract patterns by feeding large amounts of data through successive networks of artificial neurons, and refining the way those networks respond to the input. A DL model refers to a summarized relationship or structured training data set created by applying a DL algorithm to certain data, such as image data. In one example, analyzing images with the same identifier through a DL algorithm can produce a DL model. A number of DL models from various institutions can be processed to produce a federated DL algorithm that is used to improve image analysis.

Advances in DL have allowed systems to recognize objects in photographs using massive open source data sets including images tagged by large communities over the Internet. However, in the medical field, limited resources have created a barrier for tagging images and training DL algorithms.

To overcome the logistics associated with transferring data, the present invention contemplates that a number of DL models can be collected and processed for clinical use. The current system can incorporate image data and/or non-image data of a patient—vital signs, lab values, medication, historical information, and clinical reports—to develop DL models that can be used in the healthcare environment.

For example, a radiologist reviewing a chest CT may be prompted to evaluate a pulmonary nodule in a specific region of the lung. Alternatively, in certain embodiments, eye tracking may signal a region of interest that requires additional automated image processing to determine the presence and/or clinical significance of a pathologic finding, such as a pulmonary nodule. Such a computer-user interaction might involve additional computer commands, such as voice commands, that prompt the initiation of computerized image analysis.

One objective of the current invention is to provide a system that is configurable to receive and combine one or more inputs to quickly and accurately produce a report.

Certain preferred embodiments of the present invention may utilize eye tracking technology to identify the instantaneous and precise ROI in a medical image or within a set of medical images corresponding to one or more findings transmitted to the system by the reader speaking or inputting certain medical data. The system then utilizes a matching algorithm for matching the one or more inputs—such as, recorded observations or findings—to one or more templates from a set of templates.

Another objective of the present invention is to facilitate the preparation of a report using certain features of an image or a ROI that can serve as inputs into a matching algorithm or automatically link to a template from a set of structured data elements, thereby improving accuracy and reliability of the output—that is, the report.

An additional objective of the present invention is to facilitate production of a report while simultaneously tagging or categorizing an image or a ROI. Specifically, the current invention facilitates the use of eye tracking to record gaze data and associate the gaze data with terms from the report. The system can automatically process an image in order to identify a ROI within the image for training a machine learning algorithm.

While examples from the medical industry are used to describe many embodiments of the present invention, the present invention can be used in a number of industries.

Specifically, the current invention provides an opportunity to combine audio data, image and/or ROI data, and structured data schemas to train algorithms that can improve the accuracy, efficiency, and clinical utility of medical image analysis and reporting. These algorithms can be trained in either a supervised or unsupervised mode using data sets derived from the simultaneous collection of input data—such as, eye tracking and speech utterance data.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

FIG. 1 illustrates a flowchart for producing a report by matching an utterance to a template, according to one preferred embodiment of the present invention;

FIG. 2 illustrates a flowchart for producing a report by matching a ROI to a template, according to another preferred embodiment of the present invention;

FIG. 3 illustrates a flowchart for producing a report by matching at least one input to a template from a set of templates, according to an added preferred embodiment of the present invention;

FIG. 4 illustrates a flowchart for displaying an information component and producing a report using one or more inputs, according to an additional preferred embodiment of the present invention;

FIG. 5A illustrates a flowchart for processing a plurality of DL models to produce a single DL algorithm, according to another preferred embodiment of the present invention;

FIG. 5B illustrates a flowchart for outputting a report, according to an additional preferred embodiment of the present invention.

FIG. 6 illustrates a flowchart for processing a medical image through a trained DL algorithm to produce a report, according to another preferred embodiment of the present invention;

FIG. 7 is a diagram illustrating an exemplary operation for imaging analysis and reporting, according to an embodiment of the invention;

FIG. 8 illustrates an exemplary report that is produced using certain features of gaze data as inputs, according to an embodiment of the invention;

FIG. 9 illustrates a narrative template of a set of templates, according to an embodiment of the invention;

FIG. 10 illustrates a report template of a set of templates, according to an embodiment of the invention;

FIG. 11 illustrates a report template of a set of templates, according to an embodiment of the invention;

FIG. 12 illustrates an exemplary report output, according to an embodiment of the invention;

FIG. 13 is an exemplary computing system that may implement all or a portion of the invention;

FIG. 14 is an exemplary cloud computing system that may implement all or a portion of the invention; and

FIG. 15A is an exemplary Deep Learning neural network, according to an embodiment of the invention.

FIG. 15B is an exemplary Deep Learning system, according to an embodiment of the invention.

### DETAILED DESCRIPTION

The present invention relates generally to a system and methods by which at least one input from a user may be analyzed to identify objects, recognize speech, and categorize an image or a ROI in order to produce a report. Certain preferred embodiments of the present invention relate to a system that is configurable to receive a variety of inputs such that a user may choose the images, information, data, or other content for review by the user, the information that will result from that review and be inputted into the system, and the type of report that may be generated.

As described in further detail below, certain preferred embodiments of the present invention may utilize an algorithm to correlate a user's verbalization (the user's “utterance”), the image data, and/or the ROI data to one or more terms within a set of structured data elements. In addition, the algorithm may select nodes—specifically data elements or in certain embodiments coded data elements—of the hierarchical structure to populate a template.

In certain embodiments, a user can review and/or edit a template output by the system and confirm or select the template best matching the user's observations or findings.

The current system can also assign one or more terms from the selected template to an image and/or a ROI and store the image in an identified image database to train algorithms that can enhance medical image analysis and reporting.

One of the advantages of this reporting workflow is that a user, such as a radiologist, may record observations without looking away from the image or even a specific region on the image. The radiologist can simply examine a selected image and speak and the system matches the analyzed data from at least one of the inputs—the image, the utterance, the ROI, or combinations of each—to terms in the various narrative templates or report templates. The system can then automatically select, or output for a user to select, one or more templates to produce a report.

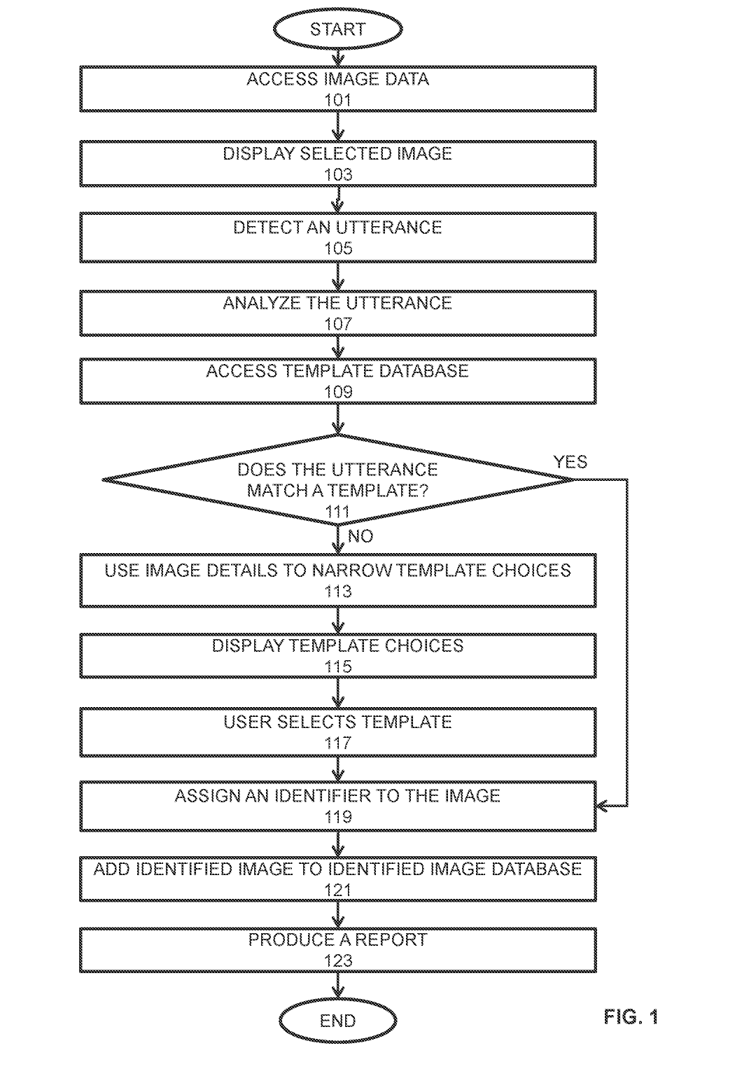

FIG. 1 illustrates a flowchart for producing a report using an utterance according to one preferred embodiment of the present invention. In operation block 101, the system accesses image data. The image data may contain information about one or more images or sets of images. For example, the image data may include details such as metadata that describes the type of image and what is being represented by the image. In operation block 103, a selected image is displayed on a display device. In operation 105, an utterance—that is, one or more recorded sounds—is detected by the system. In operation block 107, the utterance is analyzed. In decision block 109, the system accesses a template database—such as, from the main or secondary memory. In decision block 111, the system will determine whether the utterance matches a template stored in the database.

If the utterance does not match a template, in operation block 113, the system may use the image data to narrow the choices of templates that can be matched. In operation block 115, the template choices may be displayed to a user. In operation block 117, the user may select a template—such as a narrative or report template.

If, at decision block 109, the utterance matches a template from a set of templates, or once the user selects a template, in operation block 119, the system will assign an identifier to the image. In certain embodiments of the system, the identifier assigned to the image may be the template that is matched or selected. In operation block 121, the system will add the identified image to an identified image database. In operation block 123, the system will produce a report. The report may include the identified image with the matched template or the selected template.

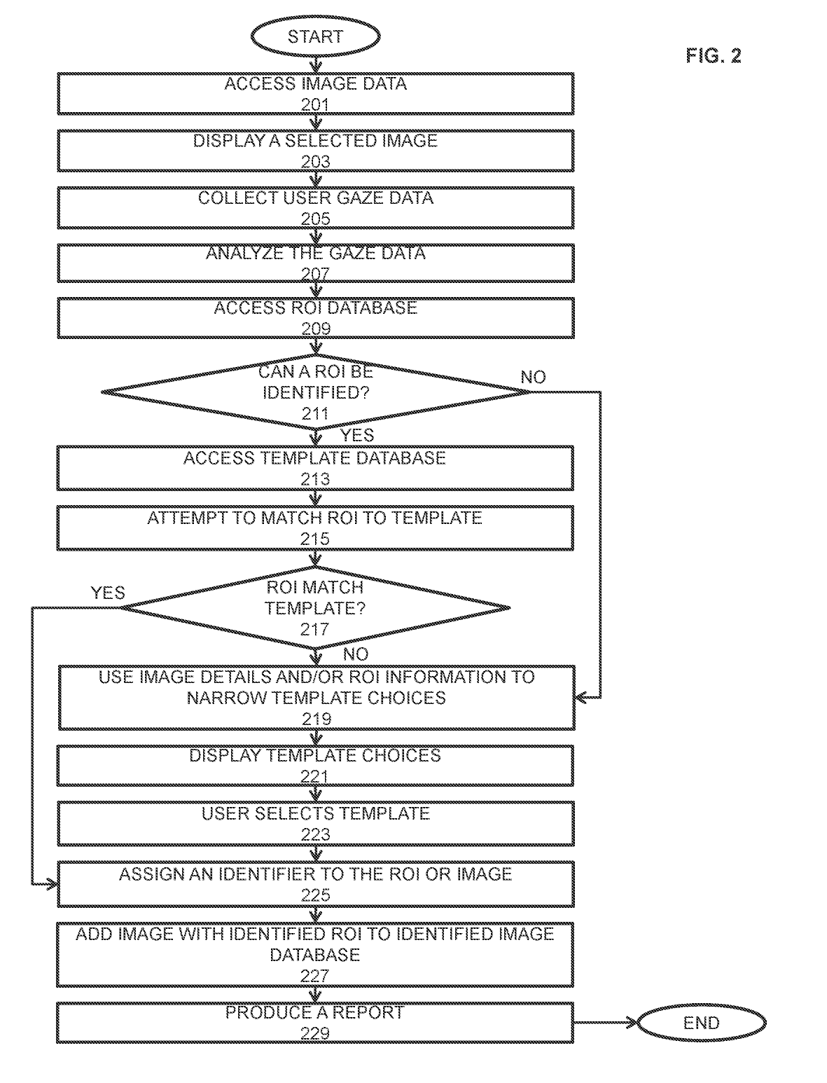

FIG. 2 illustrates another preferred embodiment of the present invention/ The flowchart illustrates the operation of a system for producing a report using information obtained from the analysis of a user's gaze. In operation block 201, image data regarding one or more images is accessed by the system. In operation block 203, a selected image is displayed on a display device. In operation block 205, the system will collect user gaze data—such as gaze coordinates or velocity of eye movements. In operation block 207, the system will analyze the gaze data. In certain embodiments, the system may use the gaze data to identify a ROI. In operation block 209, the system will access a ROI database that may include a number of ROI entries. In decision block 211, the system will determine whether matching the gaze data with the ROI database entries can identify the ROI. Alternatively, the system may use the gaze data to establish an ROI not found in the database.

If the ROI is identified at decision block 211, the illustrated system will access a template database in operation block 213. In operation block 215, the system will attempt to match the ROI to a template. In decision block 217, the system will determine whether the ROI matches a template in the template database. If no template is matched, or if the ROI was not identified at decision block 211, in operation block 219 the system will use the image details and/or ROI information from the ROI database to narrow the template choices from the set of templates stored in the template database. In operation block 221, the system will display the template choices to the user. In operation block 223, the user will select a template.

If the ROI matches a template, in operation block 225, the system will assign an identifier to the ROI. The system can assign the identifier to the ROI database entry or create a new database entry including the gaze data, ROI, and assigned identifier. In operation block 227, the system will add the selected image including the identifier to an identified image database. In operation block 229, the system will produce a report that can be communicated to the user.

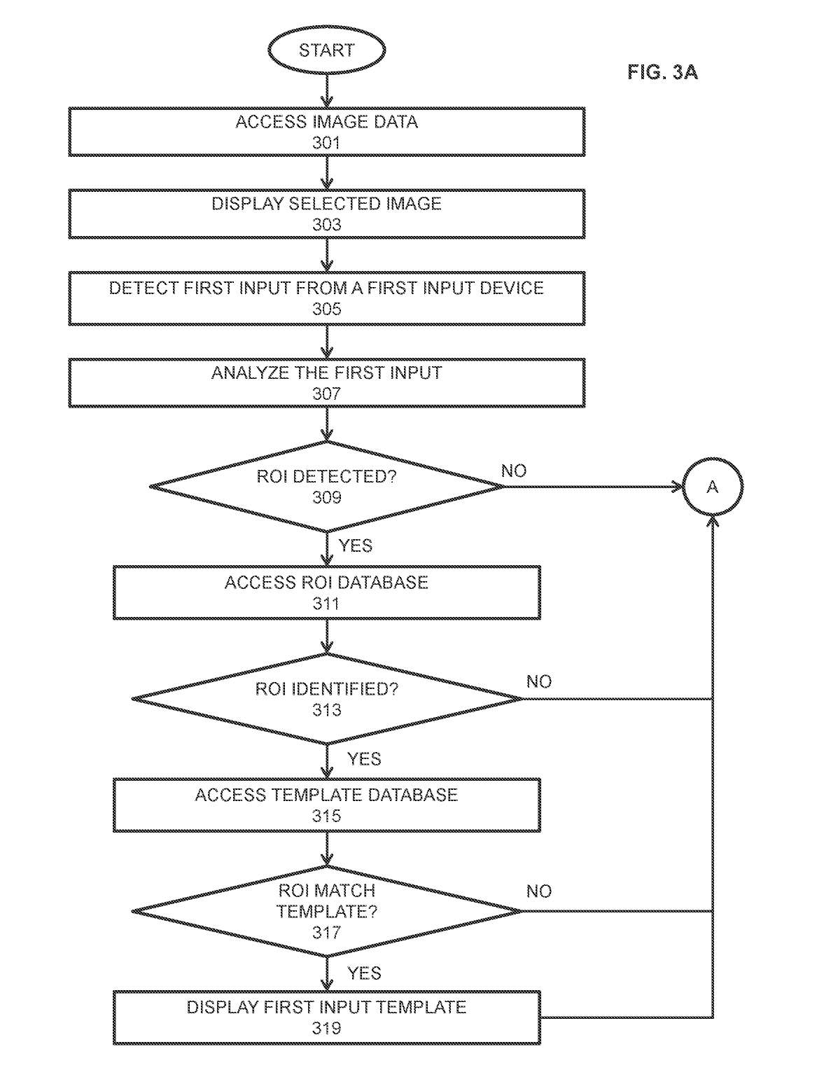

FIG. 3 illustrates one preferred embodiment for producing a report more quickly and efficiently by matching at least one input to a temple from a set of templates. In operation block 301, the system accesses image data including one or more images—such as, from a main or secondary memory. In operation block 303, one or more selected images are displayed on a display device. In operation block 305, the system detects an input from a first input device. In decision block 307, the system analyzes the first input. In decision block 309, the system determines whether a ROI is detected using data from the first input. The ROI may be detected using one or more inputs received by the system. For example, the system may use gaze data to detect a ROI or automatically link a gesture to a ROI. Alternatively, the system can use certain features of the image data to detect the ROI.

If, at decision block 309, the ROI is detected, in operation block 311, the system can access a ROI database. In decision block 313, the system can determine whether the ROI is identified. If the ROI is identified at decision block 313, the system will access a template database 315. In decision block 317, the system will determine whether the ROI data matches a template. The first input template may present the structure of a report—such as the layout or format—and may further include input fields that can be populated by information associated with that report, the ROI, or information from another input received and analyzed by the system.

If the ROI is matched to a template, in operation block 319 the system will display the first input template to a user. After the first input template is displayed, or if there is no matching template, or if the ROI could not be identified, or if the ROI is not detected, in operation block 321, the system will detect a second input from a second input device—such as a microphone, a keyboard, or a mouse. In operation block 323, the system will analyze the second input from the second input device. If the template database was not accessed before, in operation block 324, the system will access the template database. In decision block 325, determine whether the input from the second input device matches a second input template. The second input template may include one or more phrases, sentences, paragraphs, or graphical representations and may include fields denoting at least one shortcut or placeholder such as a blank slot or pick-list.

If, at decision block 325, the second input matches a second input template and the ROI was matched to a first template, in operation block 335, the system will prepare a report by populating the first input template with data elements of the second input template. In operation block 337, the system can assign an identifier to the ROI using one or more terms from the prepared report. In operation block 339, the system will add the image with the identified ROI to an identified image database. In operation block 341, the system will produce a report that can be communicated to the user.

If, at decision block 325, the second input does not match a second template from the template database, in operation block 327, the system will use the image details as inputs to narrow the incomplete or ambiguous template choices. Also, if the ROI was identified, in operation block 329, the system further narrows the ambiguous template choices based on certain features of the ROI that are used as inputs into the system—such as, imaging modality, image projection, anatomy, functional parameters, geometric parameters, and pathology. In operation block 331, the system will display the template choices and, in operation block 333, the user selects a template. In operation block 335, the system may prepare a report using the first input template, the second input template, the user's selected template, or combinations of each. In operation block 337, the system will assign an identifier to the ROI based on the prepared report and, in operation block 341, display the report to the user.

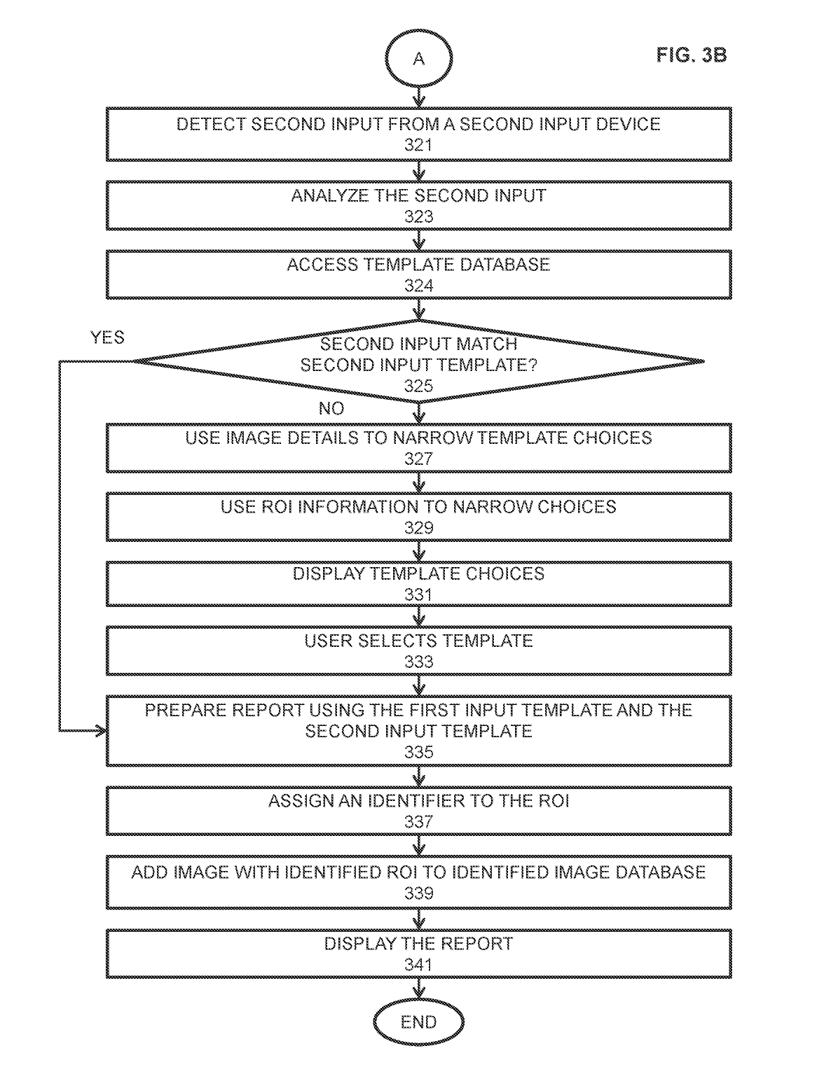

FIG. 4 illustrates another preferred embodiment of the present invention for using gaze data and an utterance to assign an identifier to a ROI and display a template. In operation block 401, image data containing at least one medical image is accessed by the system. In operation block 403, a selected medical image is displayed on a display device. In operation block 405, the system collects a user's gaze data through an eye tracking device. In operation block 407 the user's gaze data is analyzed by the system. In operation block 409, a ROI database is accessed by the system. In decision block 411, the system will determine whether the ROI is detected—such as by matching a ROI database entry to the gaze data. If yes, in operation block 413, the system will identify the ROI. Alternatively, the system may use the gaze data to establish a gaze data ROI. In decision block 415, the system will determine whether the ROI is associated with an information component—such as, clinical information associated with the ROI database entry. If the ROI is associated with an information component, in operation block 417, the information component will be displayed on the image or the ROI and the operation will continue to decision block 419.

If the ROI is not associated with an information component, or if the system could not detect a ROI, in decision block 419, the system will determine whether an utterance is detected. If an utterance is detected, in operation block 421, the system will analyze the utterance. In operation block 422, the system will access a template database, and in operation block 423, match the utterance to a template from a set of templates. In operation block 425, the system will assign an identifier to the ROI using a term from the matching template. If an information component was associated with an identified ROI, in operation block 427, the system will augment the information component according to information in the matching template. If no information component was associated with the ROI, the system can create one based on the matching template and link it to the identifier. In operation block 427, the system will display a report on the display device.

If no utterance was detected and the ROI could not be detected, in operation block 431, the system will narrow the template choices based on image data—such as, image modality, image projections, anatomy, functional parameters, geometric parameters, and pathologies. In operation block 433, the system will display template choices from which the user can select. In operation block 435, the system will assign an identifier to the image.

After the system displays a report to the user or the user selects a template from the template choices displayed, in operation block 437, the system will add the image to an image database. The system can then use the image to train and validate a DL algorithm.

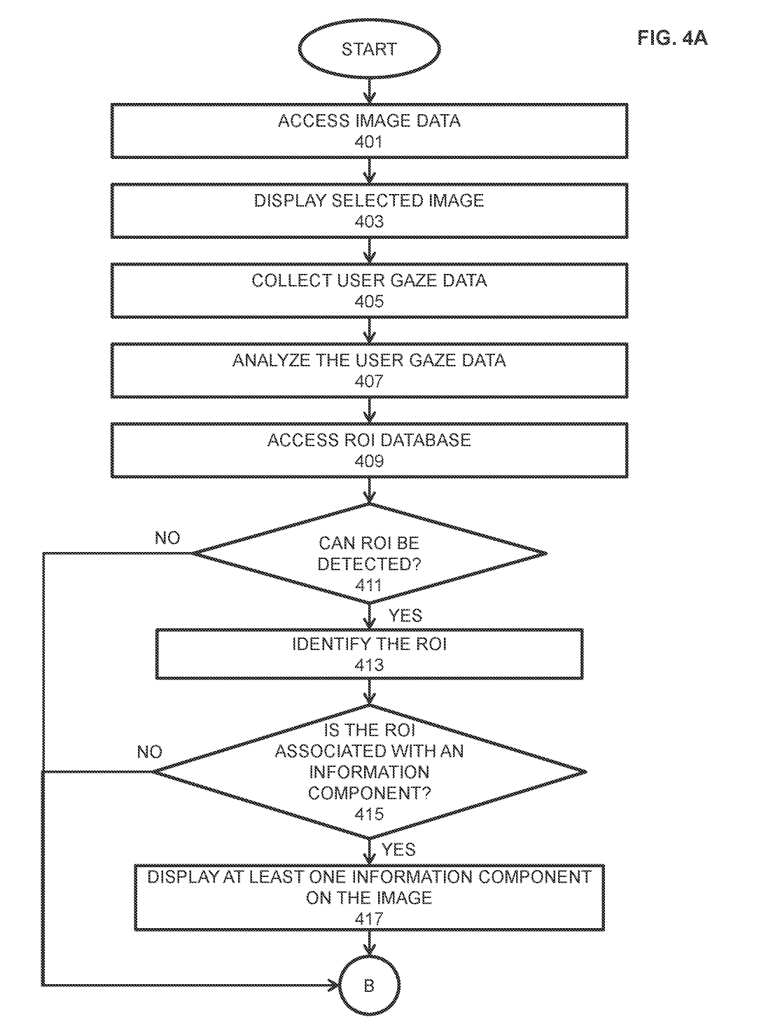

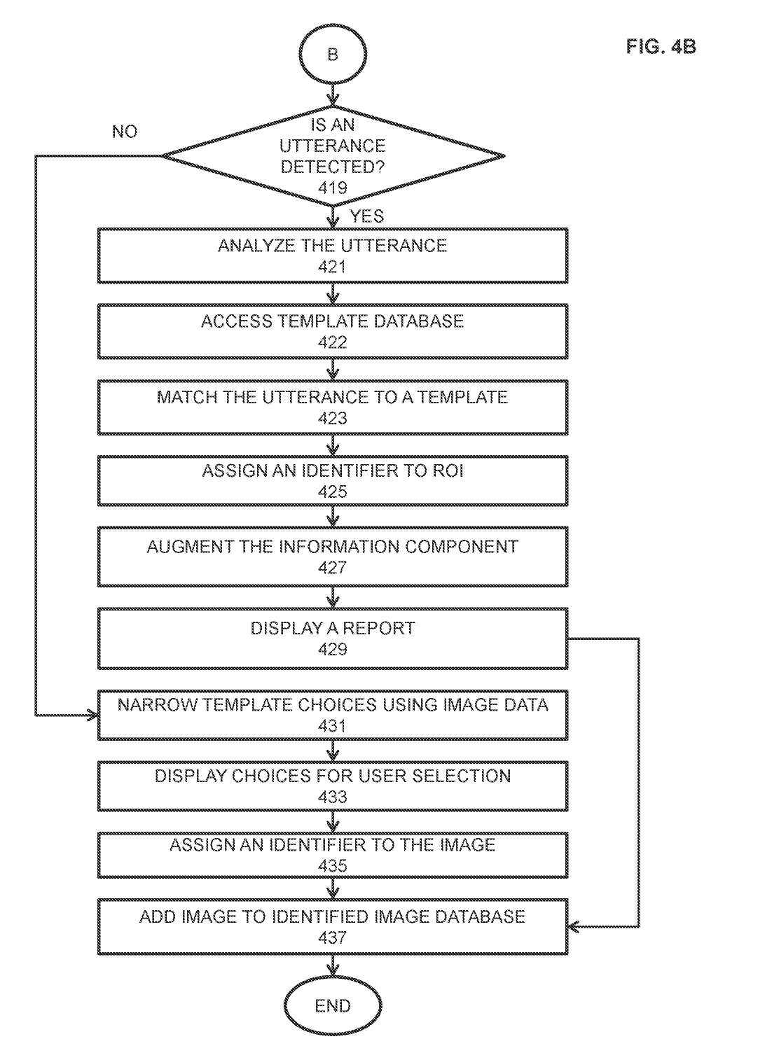

FIG. 5A illustrates a certain preferred embodiment of an operation of the system for processing one or more images using a DL algorithm. In operation block 501A, the system can access an identified image database including one or more identified images—such as a database stored in a memory of a server. In operation block 503A, the system may select an image with an identifier or a number of images with the same identifier from the database. In operation block 505A, the system will process the images using a DL algorithm. In operation block 507A, a DL model will be produced—such as, a convolution DL model. The DL model will represent a summarized relationship or structured training data produced from identifiers in the identified image database. In operation block 509A, the system may store one or more DL model in a DL learning database containing a plurality of DL models.

In operation block 511A, the system may access the DL learning database. In operation block 513A, the system will locate the plurality of DL models. In operation block 515A, the system will process the plurality of DL models to produce a single DL algorithm to enhance medical imaging analysis.

FIG. 5B illustrates a certain preferred embodiment of the system for outputting structured data elements using one or more inputs. In operation block 501B, the system can access an input database including, for example, image data, gaze data, ROI data, and utterance data—that is, speech-derived text. In operation block 503B, the system can obtain the input data.

In certain preferred embodiments of the invention, the system can use the gaze data to identify ROIs that can be used as inputs into the DL neural network along with simultaneous utterance data to produce a report. Alternatively, the system may use the gaze data to identify ROIs that correlate with structured data entered directly into the system, or indirectly using utterance data. If the ROI correlates with structured data elements, the image data can be used to train and validate a DL neural network.

In certain preferred embodiments, the DL neural network is a convolutional neural network that includes multiple layers, each layer including one or more filters that are applied to the input data. The multiple layers include at least a convolution layer, a pooling layer, and a connectivity layer. The convolution layer performs a convolution, for each of one or more filters in the convolution layer, of the filter over the input data. The pooling layer takes a portion of input data from the convolution layer and sub-samples the portion of input data to produce a single output. The connectivity layer takes all the neurons from the previous layer in the convolutional neural network and connects them to every neuron in the connectivity layer.

In operation block 505B, the system may process certain portions of the input data using one or more learnable filters to extract features from the input data. The portion of input data is analyzed by the system through a set of convolutional layers, which are cascaded so that the input of one convolutional neural network layer can be based on the output of another convolutional layer. A feature of one convolutional layer can also be used by another convolutional neural network layer. Portions of input data that the system determines do not match a structured data element are not forwarded to another convolutional layer. The input data identified by the last of the convolutional neural network layers are indications of detected structured data elements.

In operation block 507B, the convolutional neural network layers identify structured data elements in the input data. In operation block 509B, the system can output a report which includes anatomical features and/or pathologies identified in the input data. The output report can take various forms as discussed above.

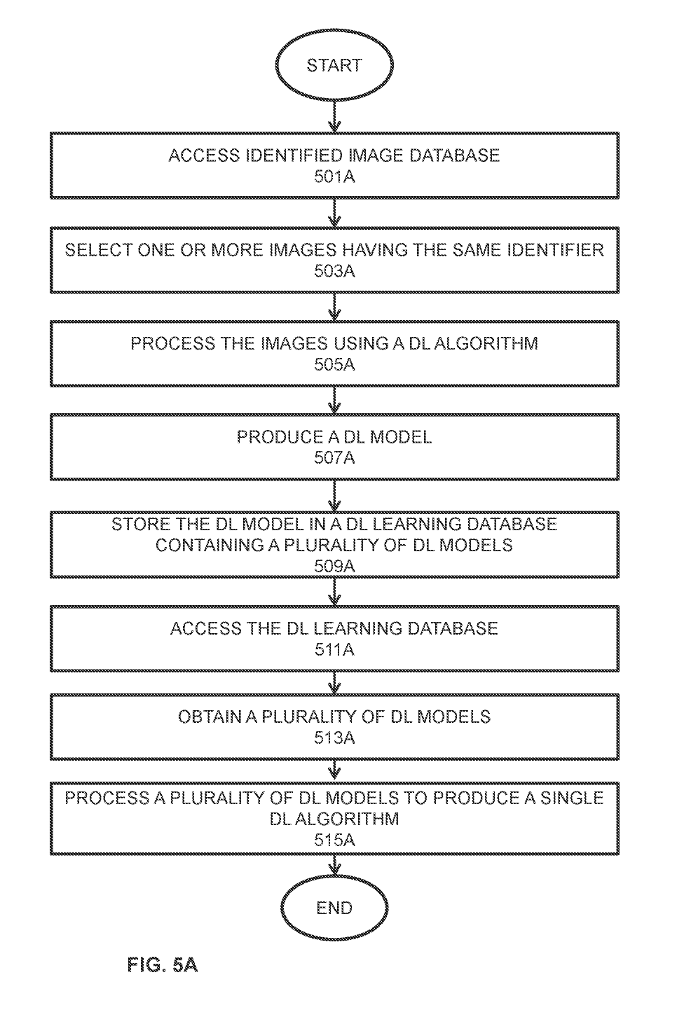

FIG. 6 illustrates a certain preferred embodiment the system for outputting a medical report. In operation block 601, the system will access image data containing one or more medical images. In operation block 603, an image is selected from the image data set. In operation block 605, the system will process the selected image using a trained DL algorithm. In operation block 607, the system will detect and automatically identify at least one medical condition and/or feature in the selected image. In operation block 609, the system will classify the medical condition and/or feature. The system may automatically classify the medical condition and/or feature using an identifier that was previously assigned to the subject area. In operation block 611, the system will output a report corresponding to the classified medical condition identified in the selected image.

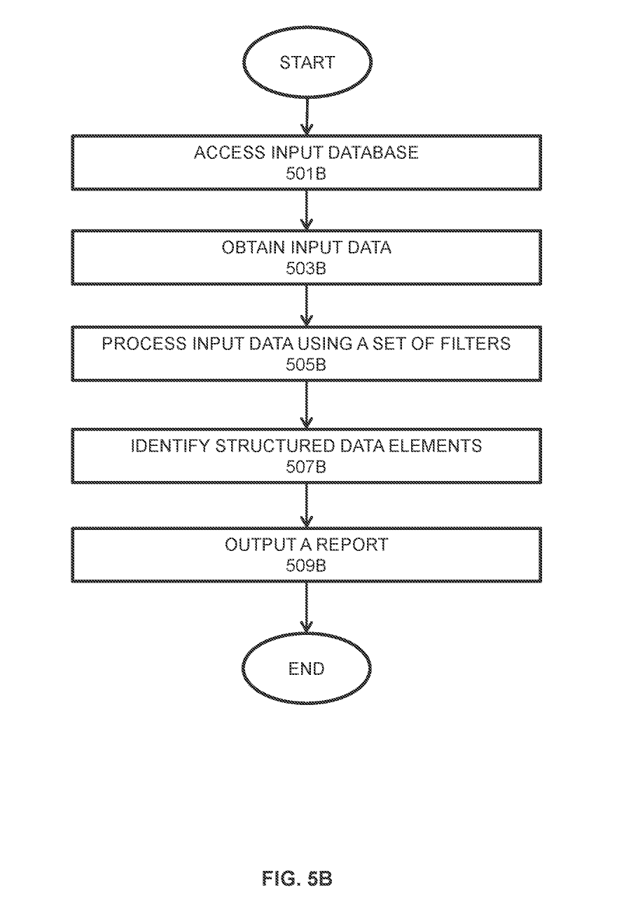

FIG. 7 illustrates an exemplary operation 700 of one preferred embodiment of the invention. Operation 700 of the system includes an eye tracking device 703 and a microphone 705. As the user 701 focuses on an image 707, the system analyzes gaze data—using gaze points 711 detected by the eye tracking device 705—to identify a ROI 709. The system continues this operation until the microphone 705 detects an utterance, which is transcribed into recorded text 713 using speech recognition software. In certain embodiments, the system may also use the image 707 data and/or the ROI 709 data to improve speech recognition software and/or narrow the templates stored in a memory (not shown). The system can then use a matching algorithm 715 to process the information derived from the utterance, the ROI 709, and/or the image 707 to output a template 717 based on matching the one or more inputs to terms in a set of structured data elements. In addition, the system may assign one or more terms of the selected template 717 to the ROI 709 as an identifier that can be used to produce a DL model. In some embodiments, the DL model can then be used as an input to train a DL neural network.

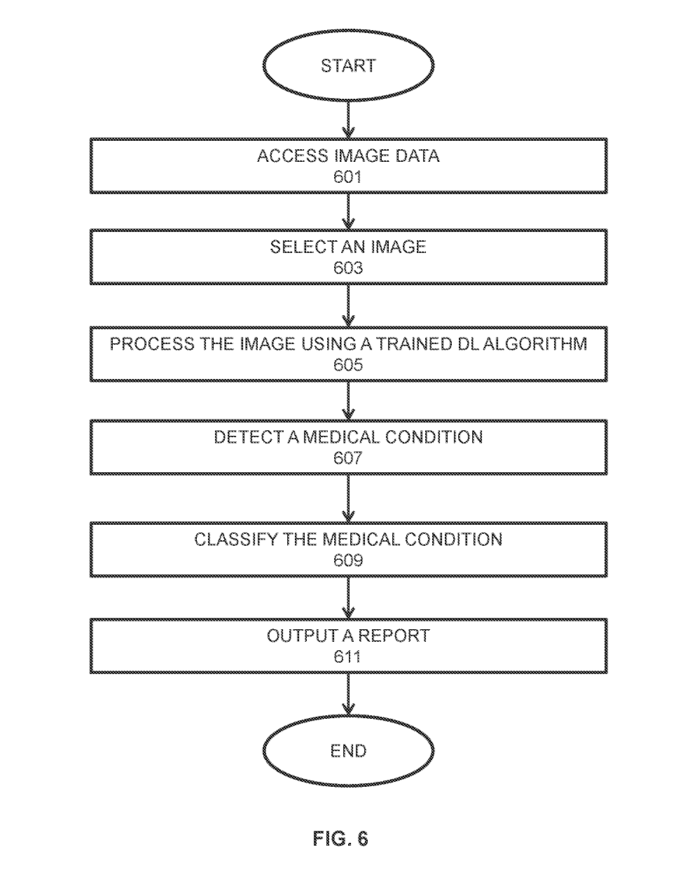

FIG. 8 illustrates the use of image and gaze data recorded by an eye tracking device to serve as an input into a matching algorithm to create a report. The process of reviewing images typically means that the reader is looking at them on a display device and, more particularly, focusing on a specific region of an image as one or more conclusions are reached. Eye tracking technology can identify the specific region by collecting gaze data using either a remote or head mounted eye tracking device. Specifically, eye tracking technology can record a user's point of gaze and movement on a 2D screen or in 3D environments based on corneal reflections.

In certain preferred embodiments, eye tracking coordinates may provide information about image data contained in longitudinal or multimodality imaging studies. Longitudinal analysis of image data might improve accuracy of matching to comparison statement speech utterances, or highlight potential inconsistencies or missed trends. Analysis of multimodality image information, such as mitrel valve information from an echocardiography, a cardiac catheterization, and/or an MRI, may further improve diagnostic and reporting accuracy.

Other image information that the system can use in a matching algorithm may include image projection, anatomy, functional parameter, geometric parameters, and/or pathology. Imaging modality may include the contrast, window level, or image plane of a CT scan, the planar radiography, the imaging sequence of an MRI, or the 2D imaging, spectral Doppler, or color flow Doppler. The image projection may include the anterior-posterior or lateral planar orientation, and/or the axial, coronal, sagittal, multi-planar reconstruction, or curved multi-planar reconstruction tomographic plan. Certain anatomical features, such as organs or structures can also serve as the input. Functional parameters that can serve as the input include density, flow, calcification, metabolism, and timing. In addition, geometric parameters include length, thickness, area, shape, and volume.

FIG. 8 illustrates an echocardiogram image 800—a heart ultrasound—of a patient with endocarditis—a heart valve infection. The circled area 802 of FIG. 8 represents the cardiologist's gaze identified by the system using an eye tracking device on the echocardiogram image 800. The system may output cross-hatches 806, as seen on the image 800, to identify the ROI 804. In one example, the cardiologist may provide the utterance “Mobile 10 by 7 anterior leaflet vegetation” to report a “growth”—vegetation—while focused on the ROI 804. The utterance is an input into the system, along with the image 800, and the ROI 804. The system may use software—such as speech recognition and/or image processing—to create a text string from the one or more inputs, which may include exact matches and/or ambiguities. For instance, the system may not recognize the words “anterior” and/or “vegetation”, or may recognize the words with a low level of confidence, thereby conflicting with other potential matching words. In addition, although the utterance did not contain any information regarding the leaflet vegetation belonging to the mitral valve, the system may use the image 800 and/or the ROI 804 to identify that area.

The system may then combine the recognized text from the utterance, with data derived from the associated image 800, and the ROI 804—including the modality, view, anticipated and identified structures, and pathology. The totality of these inputs are then used in an algorithm that matches at least one term within a set of structured data elements to output the narrative template Anterior mitral valve leaflet 806 on the image 800 as seen in FIG. 8, which can be automatically selected by the system, selected by the user, and/or audibly through a speaker (not shown).

The match to structured data does not need to be precise. Eye tracking and speech to text output could be used to “localize” the subset of content relevant to the intentions of the reading physician at that moment. In such a scenario, rather than matching a specific report finding—“There is mild-moderate mitral regurgitation”, the algorithm may only localize a subset of relevant content within the structured data set—“mitral valve” or “mitral valve regurgitation”. The user interface may then present or read back to the reading physician a small menu of structured data choices that can be selected by voice command, mouse or keyboard inputs. Because the relevant data set has been significantly filtered down from all the potential data elements in a report (potentially tens of thousands) to a very small number relevant to the precise reporting context at hand (perhaps a dozen data elements or less), a compact and simple set of choices can be presented to the user in real time, potentially on the same screen as for image review. This would allow the users attention and gaze to be minimally diverted from the images and the task of image review and interpretation.

In certain embodiments of the present invention, the temporal axis of gaze data is used in the matching process. Recently examined ROIs may be particularly relevant—as determined by eye-motion dwell or pointing, recent utterance and recently recorded data from those utterances, which can be relevant when processing the current utterance. For example, in the current embodiment, the user might have said “anterior leaflet vegetation” (deep learning supplied “mitral valve”) and then in a subsequent utterance “mobile 10 by 7”, the results from processing of the prior utterance influences the processing of the latter.

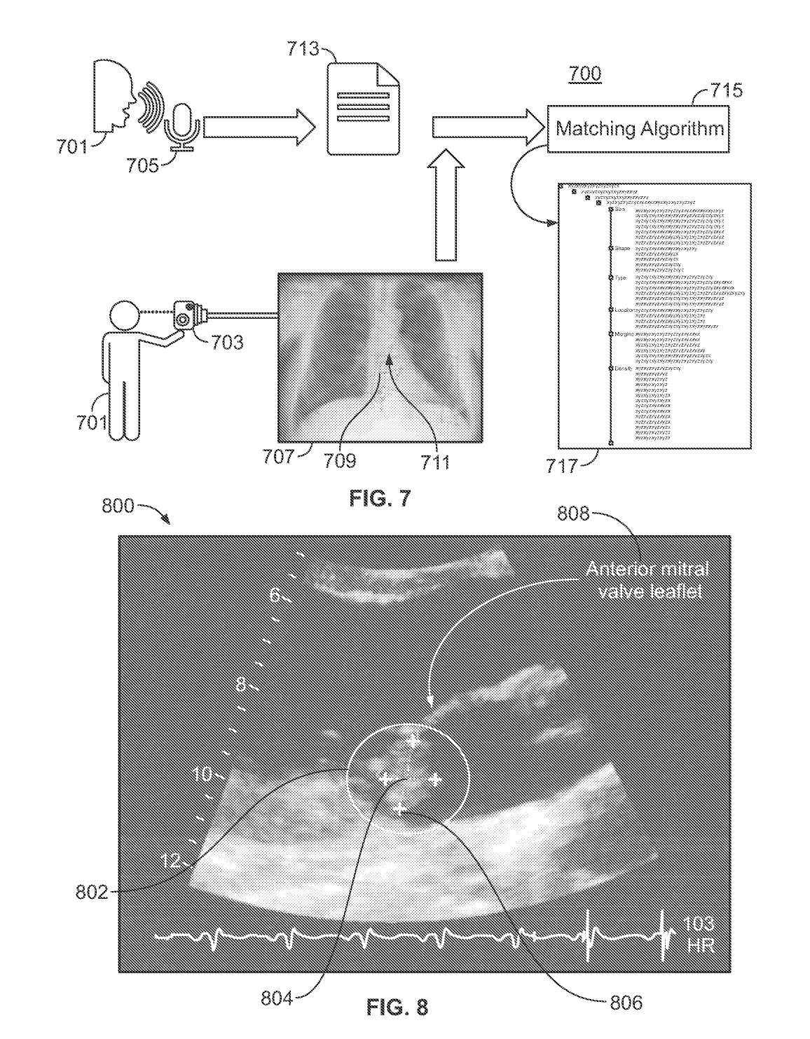

FIG. 9 illustrates an embodiment of a narrative template 900 of a set of structured data elements according to the present invention. As shown, the template 900 includes a hierarchy of terms for the [SIZE] field 902 and [SHAPE] field 904 and a sub-hierarchy of terms for the [LOCATION] field 906, [MARGINS] field 908 and [DENSITY] field 910. A user such as a radiologist might select the narrative template 900 shown in FIG. 9 by gazing at a region associated with a liver displayed on an image—such as an abdominal MRI scan—while uttering “small mass invading the liver round more dense than fat”. The system can analyze the image data and the user's gaze data, identify the ROI, and process the words in this utterance through a matching algorithm to populate and record a matching template associated with the inputs. More precisely, these inputs are matched to corresponding terms of the set of structured data elements including any sub-hierarchies of the template 900, which may be narrowed by the image data and/or ROI data, yielding the following data elements:

[PATHOLOGY] 900: mass 951

[SIZE] 902: small 932

[SHAPE] 904: round 941

[LOCATION] 906:
[RELATIONSHIP] 921: invading 953
[ORGAN] 922: liver 966

[MARGINS] 908:
[TYPE] 924: blank

[DENSITY] 910:
[COMPARISON] 926: more dense than 981
[MATERIAL] 927: fat 991

It should be noted that the [MARGINS] field 908 returned no matches and is left blank.

In certain preferred embodiments, the system will display ambiguous matches such that the user can select a template. The system may also determine the matching template independent of word form or word order of the utterance such that the utterance “round mass small more dense than fat invading the liver” yields the same result.

In certain embodiments, the system may analyze the image and the user's gaze to determine that one or more fields, such as the [LOCATION] field 906, matches a template. For example, the system will match data derived from a radiologist gazing at the liver on an image and uttering “round mass small more dense than fat invading” to corresponding terms of the set of structured data elements to yield the same results as above. Specifically, the gaze data may be used to identify the ROI such that the system can match the term “liver” to a data element in a database associated with the ROI.

The data elements corresponding to the terms 932, 941, 953, 966, 981, 991 are used to populate the template such that the utterance, the ROI, the image, or combinations of each, match to the populated template. The populated template is determined to match to the recorded inputs to generate the narrative template “The patient has a small 932 round 941 mass 950 invading 953 the liver 966 that is more dense 981 than fat 991”. The narrative template 900 may then be communicated to the user—for example, audibly through a speaker, visually through a display unit, or a combination of both.

In certain preferred embodiments, the system may use data associated with the image and/or ROI to aid in the matching process by narrowing the utterance to a certain set of words or a lexicon associated with the data. For example, the narrative template 900 of FIG. 9 may also match the following utterance: “the left kidney appears to be invaded by a mass that is spherical and large” yielding the following populated template: “The patient has a large 934 round 941 mass 950 invading 953 the left kidney 967” despite the following variations in inputs by the user: (1) the order of the words in the utterance is reversed from the order in which the terms appear in the set of structured data elements, (2) the word “invaded” is used in the utterance, while the set of structured data elements includes the term “invading”, (3) the utterance includes extraneous terms such as “appears” and “that is”, (4) the utterance fails to include information present in the template; in particular, the utterance does not contain any words relating to the [MARGINS] 908 and [DENSITY] 910 sub-hierarchies, (5) the utterance and the template use the multi-term phrase “left kidney”, (6) the word “round” is not used in the utterance, but associated with the ROI, and (7) the word spherical is not associated with the image or ROI data, and thereby not considered in the matching process.

The matching algorithm allows a user to select a template when ambiguities arise between words of the utterance, the ROI, the image, or combinations of each and the terms within the set of structured data elements including sub-hierarchies. In addition, the matching algorithm considers the structure, semantics, context, and content of the template through each sub-hierarchy, and in particular, the matching algorithm accounts for the number of terms that can be selected in a given sub-hierarchy. In FIG. 9, for instance, the [RELATIONSHIP] sub-hierarchy 921 allows the selection of exactly one item (single-valued), while the [SIZE] hierarchy 902 allows the selection of zero or one item (nullable single-valued) and the [TYPE] sub-hierarchy 924 allows the selection of multiple terms (multi-valued).

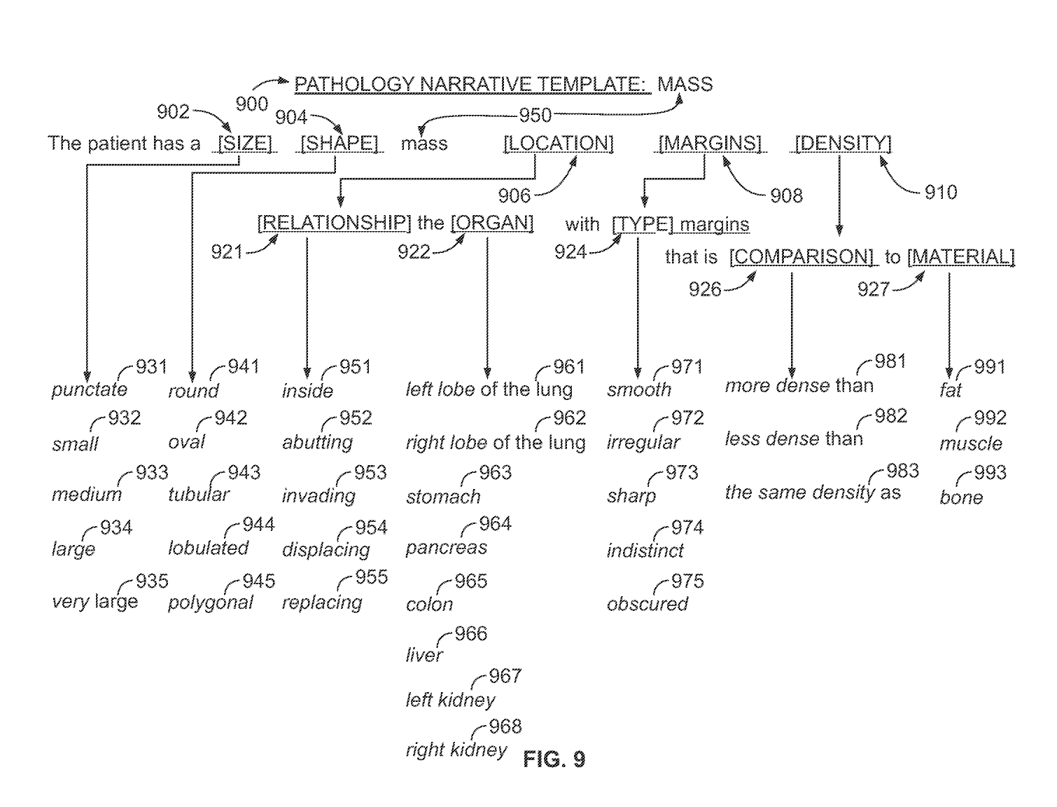

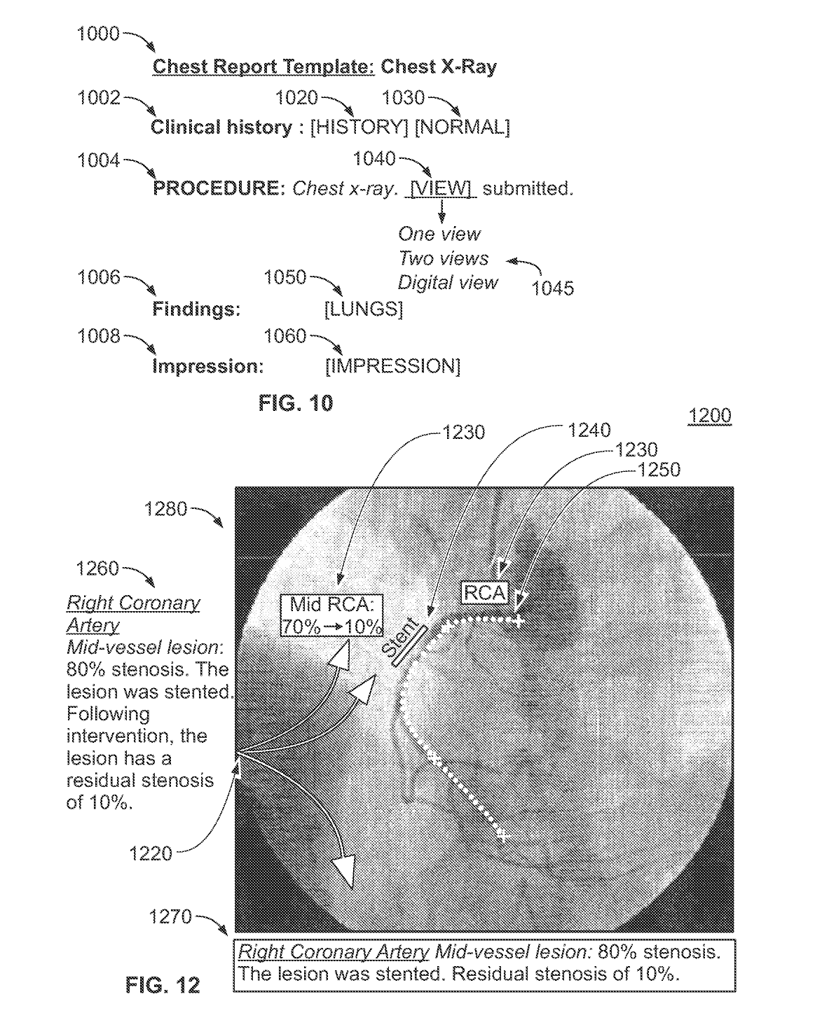

FIG. 10 illustrates an embodiment of a report template 1000 and FIG. 11 illustrates an embodiment of a set of narrative templates 1100 placed within the report template 1000 of FIG. 10 according to the present invention. More specifically, FIG. 10 illustrates a report template 1000 for a chest x-ray procedure, including support for variations in the number/type of views acquired and placeholders for additional narrative templates regarding clinical history 1002, procedure 1004, findings 1006, and impression 1008. FIG. 11 is one example of the narrative templates 1100 used to populate the report template 1000, where the bracketed fields 1020, 1050, 1060 denote where the associated narrative templates 1100 should be placed within the report template 1000.

A user such as a radiologist might invoke the report template 1000 illustrated in FIG. 10 by selecting a two-view x-ray image of a patient's chest and gazing at the patient's lungs. Based on the selected image and the identified ROI—the patient's lungs—, any words detected by the system are narrowed by the context of the image and ROI. For example, the system may limit the speech recognition to a certain lexicon and/or reduce the number of terms in the set of structured data elements that can match an utterance. The system then obtains the “chest x-ray” procedural report template 1000 and populates the [VIEW] field 1040 with the terms “two views” 1045 based on the selected image. The radiologist may complete the report template 1000 with the following series of utterances:
“history asthma”
“clear well-inflated”
“no mass”
“no pleural effusions”
“negative chest”
   which are then matched to the associated narrative templates 1100 shown in FIG. 11. Matching these utterances and the ROI to narrative templates 1100 associates the words of the utterances and the ROI to the terms of the set of structured data elements, which are represented by data elements:

[HISTORY] 1020: Asthma 1121

[LUNGS] 1050:
[STATUS] 1130: clear 1131
   well-inflated 1132
   no mass or adenopathy is identified 1183
no pleural effusions 1184
[IMPRESSION] 260: Negative chest x-ray. 1161
   The system can then select terms along with the corresponding data elements of the set of structured data elements to populate the narrative templates 1100. The data elements assist with positioning the narrative templates 1100 in the correct positions within the report template 1000:
Clinical history 1002: Asthma 1121.
Procedure 1004: Chest x-ray. Two views 1045 submitted.
Findings 1006: The lungs are clear 1131 and well-inflated 1132. No mass or adenopathy is identified 1183. No pleural effusions 1184.
Impression 208: Negative chest x-ray 1161.

Alternatively, a radiologist could retrieve the report template using the utterance “normal two view chest x-ray”, which the algorithm matches to the report template 1000 in FIG. 10, including populating the findings 1006 specified by the [NORMAL] field 1030. The normal template includes the terms of the set of structured data elements denoted by “*” in FIG. 11 such that the report template 1000 is:
Clinical history 1002: [HISTORY] 1020
Procedure 1004: Chest x-ray. Two views 1045 submitted.
Findings 1006: The lungs are clear 1131 and well-inflated 1132. No mass or adenopathy is identified 1183. No pleural effusions 1184.
Impression 208: Negative chest x-ray 1161.
   Deviations from the normal template may then be specified by gazing at the patient's lungs on the image and the following series of utterances:
“history asthma”
“mildly prominent markings”
“no mucus plugging”
“no acute abnormality”
   which are matched to the associated narrative templates 1100, wherein the data elements associated to the terms of the set of structured data elements populate the report 1000:
Clinical history 202: Cough 1122.
Procedure 204: Chest x-ray. Two views 1045 submitted.
Findings 206: The lungs are clear 1131 and well-inflated 1132.
There is no evidence of mucous plugging 1185. There is mild 1141 prominence and coarsening of the lung 1186. No mass or adenopathy is identified 1183. No pleural effusions 1184.
Impression 208: No acute abnormality 1162.
   The data elements are used to automatically position these new findings in the report template 1000 and to automatically replace the [IMPRESSION] 1060 “Negative chest x-ray” 1161 with the [IMPRESSION] 1060 “No acute abnormality” 1162. The system may then visually and/or audibly communicate the report template to the.

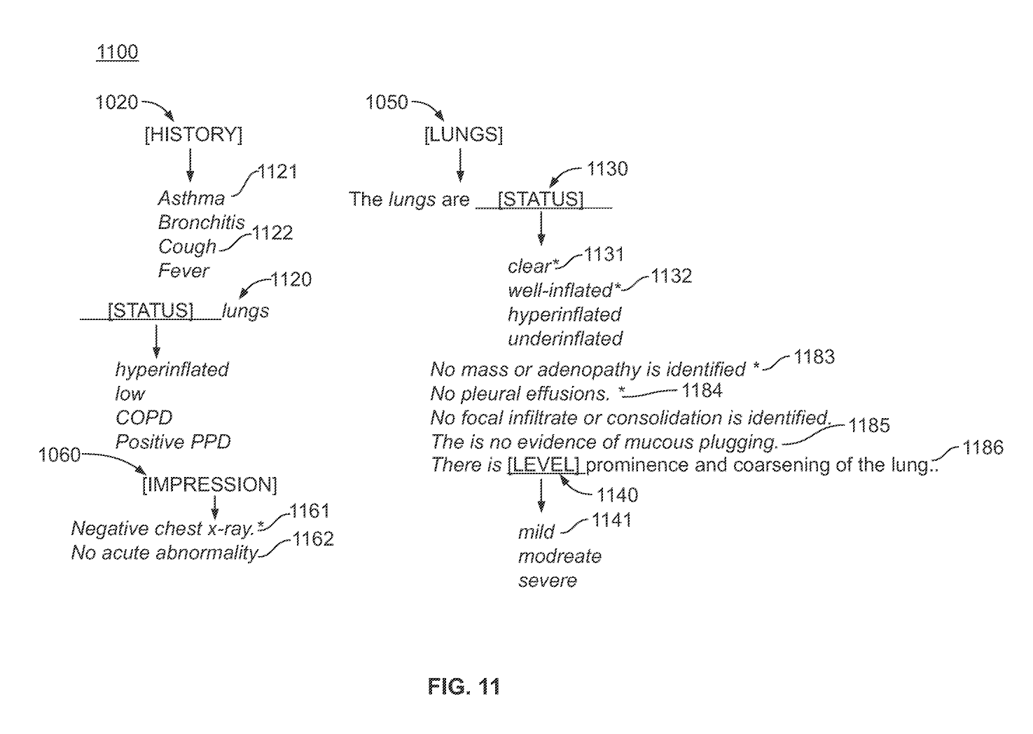

FIG. 12 illustrates an exemplary output 1200 in accordance with an embodiment of the invention that associates certain information and data (the “association” illustrated in FIG. 12 as a set of arrows 1220) of a narrative template 1260. In addition, the FIG. 12 illustrates the displayed narrative template 1260 and various information components 1230 on a medical image 1280.

Embodiments of the invention may detect an anatomical feature in the image 1280, or that which the user is gazing at, to identify a ROI 1250. The embodiment shown in FIG. 12 emphasizes the ROI 1250 by a dashed line and “+” signs positioned at the termini of the line to produce a line design and its position to illustrate the path of the artery that is the subject of the user's gaze.

Embodiments of the system may allow a user to select a narrative template regarding the anatomy and/or pathologies recorded during review or examination of the one or more selected images to produce or augment the associated narrative template. With respect to the image shown in FIG. 12, the narrative template 1260 provides information recorded about a mid-RCA stenosis and its treatment. In certain preferred embodiments, the narrative template 1260 may be automatically generated from a set of structured data elements based on the image 1280, the utterance, the ROI 1250, or combinations of each.

Certain embodiments of the present invention may use a reference frame to automatically match the image 1280, the ROI 1250, or a combination of both to at least one term of a template, such as a report template or the narrative template 1260, and generate data information components 1230 (callouts, icons, captions, etc.) on the image based on the terms in the matching template. In some embodiments, the ROI 1250 may already be tagged with an identifier such that one or more information components 1230 are output in response to the detecting the

ROI 1250. In certain embodiments, the information components 1230 may be output in response to the image 1280, the ROI 1250, the narrative template 1260, or in combination.

Advantageously, certain embodiments of the system may include one or more data information components 1230 that provide summarized information, such as an acronym, regarding one or more anatomical features and/or pathologies under consideration, such information components including those termed a “callout” for purposes of this application. The image 1280 shown in FIG. 12 includes a data information component 1230 that outputs an acronym “RCA” that the user is viewing an image showing the right coronary artery and information from the narrative template 1260 but in summarized form regarding the anatomical feature the user is gazing at—that is, information regarding how the stenosis of the imaged artery changed following surgical intervention (“70%−>10%”).

Advantageously, certain embodiments of the system may offer to a user one or more icons that may be of relevance to that which is the subject of the user's focus and automatically position, or permit the user to select and position the one or more icons in association with a ROI. The image 1280 shown in FIG. 12 includes a graphical component 1240—in this example, an elongated rectangular shape that symbolizes a stent and informs the viewer that a stent was positioned in the artery and the location of the stent. Such an icon may graphically represent that which is expressed in words in the recorded narrative structure 1260 and/or that which is associated with the ROI 1250. In this case, the stent icon represents the words “The lesion was stented.” One or more numbers, alphabets, words, or other symbols to provide confirming or new information may accompany such icons. To illustrate, in FIG. 12, the icon symbol is accompanied by the word “stent”.

Advantageously, certain embodiments of the present invention may include an additional information component 1270—such as a caption—that adds to, summarizes, and/or highlights the narrative template 1260, the ROI 1250, or a combination of both. The embodiment of the system from which the image 1280 shown in FIG. 12 was produced includes an information component 1270 that in this example provides the medical terms of the ROI 1250 “Right Coronary Artery” and a summary of the information in the matching narrative template 1260.

In FIG. 12, the image presents in more complete form the summarized information of the data information components 1230, graphical component 1240, and information component 1270—that is, the stenosis data entered for the imaged artery and the result of the surgical intervention.

Embodiments of the system may also include additional components by which information may be entered with respect to one or more images. Depending on the embodiment of the invention, the displayed information and graphical data components may be inserted and positioned as desired manually through an input device or inserted, in part or wholly, through automation features. Depending on the embodiment of the invention, the displayed information and graphical data components that may be inserted and positioned on a selected image by a user, such as a radiologist, doing the study of the selected anatomical feature or pathology may be subsequently manipulated—for example, by the reporting or referring physician—as desired. For example, a reporting or referring physician may wish to select only certain displayed information and graphical data components shown through the image 1280 for purposes of presenting the results of the study to a patient or patient representative. The information that is not chosen by the physician to present to the patient may be, for example, historical data with which the patient is already familiar.

Embodiments of the system may also allow a user to modify the position, nature, or content of the displayed information or graphical data components through one or more inputs, and, in so doing, modify the associated narrative template. For example, a user may wish to change the information displayed in the data information component 1230—“10%” to “5%”, whereupon the system would modify the information in the narrative template 1260 which, in turn, would modify the image caption 1270 to reflect the new residual stenosis value,—“Residual stenosis of 5%”. The system may also detect a new ROI using various properties of the gaze data, such as the pattern of fixations and saccades, whereupon the system would modify the associated narrative template 1260 to reflect these changes. As an example, a user may shift their focus from the RCA to the stent graphical component 1240 and describe the diameter and length to be 5 mm in diameter and 25 mm in length, whereupon the system would modify the narrative template 1260 and caption 1270 to also describe the new diameter and length information, as in “The lesion was stented with a 5 mm (D)∓25 mm (L) stent.”

In certain preferred embodiments, the system may assign the one or more terms from a template or information component to a ROI. The assigned identifier may describe a ROI and allow it to be easily found using a basic search. In addition, certain components may be assigned an identifier, such a graphical component or information component. In certain embodiments, the identifier may be a knowledge tag that describes or defines some aspect of a ROI. Knowledge tags can be used to capture and collect certain user inputs—utterances and gaze data—for creating a profile. These profiles may further reference an information resource, such as a term from a set of structured data elements. Advantageously, knowledge tags may supplement the term by providing additional structure, semantics, content, and context to the ROI.

In the image 1280 shown in FIG. 12, the system may assign the ROI 1250 an identifier relevant to the narrative template 1260 or one or more information components 1230. For example, the identifier may be “RCA” or “Right Coronary Artery”. If the system detects the utterance “RCA”, the system, as shown in FIG. 12 can emphasizes the RCA by a dashed line and “+” signs. Advantageously, the identifier will be stored in a database of the system such that if a user or another individual gaze at the ROI 1250 at a later time, the narrative template 1260, data information components 1230, graphical component 1240, and caption 1270 will automatically be output by the system. In addition, the image 1280 and identifier can then be used to train and validate a DL algorithm.

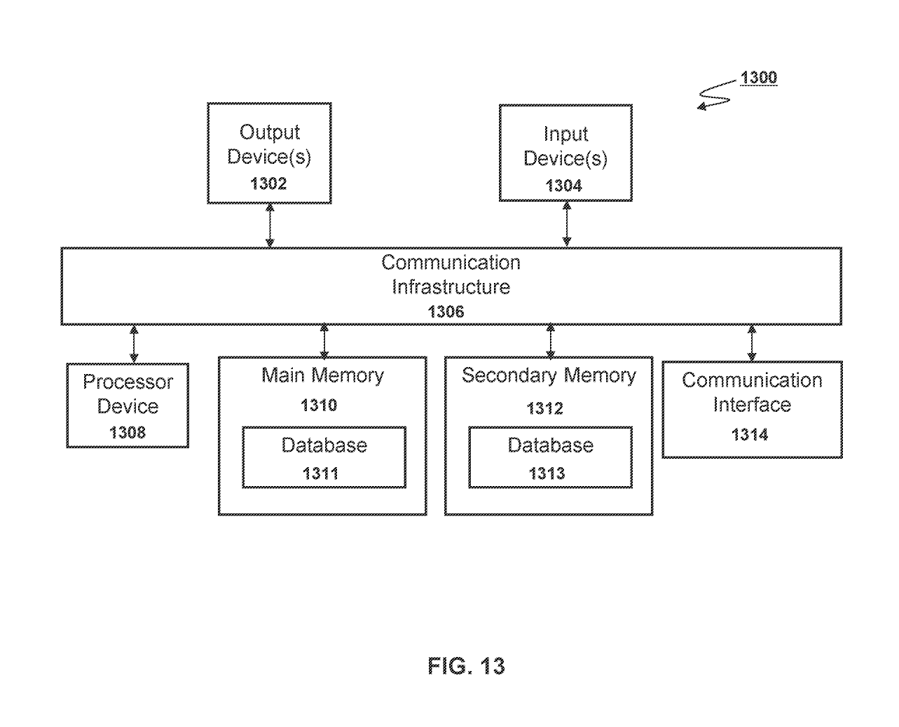

An exemplary system 1300 according to the invention is shown in FIG. 13. The exemplary system 1300 as shown may be used to implement the methods according to the invention using one or more processor devices 1308. The system 1300 includes at least one output device 1302 and at least one input device 1304 connected to a communication infrastructure 1306—such as a bus—, which forwards data from the communication infrastructure 1306 to other components of the system 1300.

Examples of output devices 1302 may include a display device and speakers. The display device may be, for example, a monitor, touch screen, or any other computer peripheral device capable of entering and/or viewing data. It is also contemplated the display device may be a web-based interface accessible through the system 1300. According to the invention, the system 1300 may be a small-sized computer device including, for example, a personal digital assistant (PDA), smart hand-held computing device, cellular telephone, or a laptop or netbook computer, hand held console, tablet, or similar hand held computer device, such as an iPad®, iPad Touch® or iPhone®. In some embodiments, the display device can incorporate a flexible display element or curved-glass display element to conform to a desired shape.

The output device 1302 may also include one or more speakers capable of converting electronic signals into audible sound waves. The speakers can be used for reproducing sounds such as speech or music. In some embodiments, the speakers can be used to produce an alert—such as, a verification or error alert.

Examples of input devices 1304 may include microphones, touch sensors, eye tracking devices, and the like. In certain embodiments, input devices 1304 can include a natural user interface that may recognize interactions without requiring touch. For example, a natural user interface may be configured to support voice recognition to recognize particular utterances as well as to recognize a particular user that provided the utterance. In another example, a natural user interface may be configured to support recognition of gestures, presented objects, images, eye movements, and so on through use of a camera.

A microphone can include any device that converts sound waves into electronic signals. In some embodiments, a microphone can be sufficiently sensitive to provide a representation of specific words spoken by a user; in other embodiments, microphone can be usable to provide indications of general ambient sound levels without necessarily providing a high-quality electronic representation of specific sounds.

Touch sensors may include a capacitive sensor array with the ability to localize contacts to a particular point or region on the surface of the sensor and in some instances, the ability to distinguish multiple simultaneous contacts. In some embodiments, touch sensors can overlay a display device to provide a touchscreen interface for translating certain contact—taps and/or other gestures—into specific user inputs.

Eye tracking devices may be capable of measuring eye positions and eye movement. More specifically, the eye tracking device may incorporates illumination, sensors and processing to track eye movements and gaze points. The use of near-infrared light allows for accurate, continuous tracking regardless of surrounding light conditions. This technology is often referred to as pupil center corneal reflection eye tracking. The eye tracking device may be a remote eye tracking, a mobile eye tracking, or a wearable eye tracking device. It is contemplated that the eye tracking device may include a camera including a standard webcam.

Eye tracking devices can be calibrated using software, in which the user focuses on a blue dot as it moves to a variety of different locations on a display device. More specifically, the eye tracking device operates by shining infrared light into the eye of the user to create reflections that cause the pupil to appear as a bright, well-defined disc in eye tracking device. The corneal reflection is also generated by the infrared light, appearing as a small, but sharp, glint outside of the pupil. The point being looked at by the user is then triangulated from the corneal reflection and the pupil center.

The system 1300 includes one or more processor devices 1308, which may be a special purpose or a general-purpose digital signal processor device that processes certain information. The system 1300 also includes a main memory 1310 and/or secondary memory 1312. Main memory 1310 includes, for example, random access memory (RAM), read-only memory (ROM), mass storage device, or any combination thereof. Secondary memory 1312 may include, for example, a hard disk unit, a removable storage unit, or any combination. Main memory 1310 and/or secondary memory 1312 may each include a database 1311, 1313, respectively.

The system 1300 may also include a communication interface 1314, for example, a modem, a network interface (such as an Ethernet card or Ethernet cable), a communication port, a PCMCIA slot and card, wired or wireless systems (such as Wi-Fi, Bluetooth, Infrared), local area networks, wide area networks, intranets, etc.

It is contemplated that the main memory 1310, secondary memory 1312, including database 1311 and database 1313, or a combination, function as a computer usable storage medium, otherwise referred to as a computer readable storage medium, to store and/or access computer software including computer instructions. For example, computer programs or other instructions may be loaded into the system 1300 such as through a removable storage device, for example, a ZIP disk, portable flash drive, optical disk such as a CD or DVD or Blu-ray, Micro-Electro-Mechanical Systems (MEMS). Computer programs, when executed, enable the system 1300, particularly the processor device 1308, to implement the methods of the invention according to computer software including instructions. The system 1300 may perform any one of, or any combination of, the steps of any of the methods according to the invention.

Communication interface 1314 allows software, instructions and data to be transferred between the system 1300 and external devices or external networks. Software, instructions, and/or data transferred by the communication interface 1314 are typically in the form of signals that may be electronic, electromagnetic, optical or other signals capable of being sent and received by the communication interface 1314. Signals may be sent and received using wire or cable, fiber optics, a phone line, a cellular phone link, a Radio Frequency (RF) link, wireless link, or other communication channels.

The system 1300 of FIG. 13 is provided only for purposes of illustration, such that the invention is not limited to this specific embodiment. It is appreciated that a person skilled in the relevant art knows how to program and implement the invention using any computer system or network architecture.

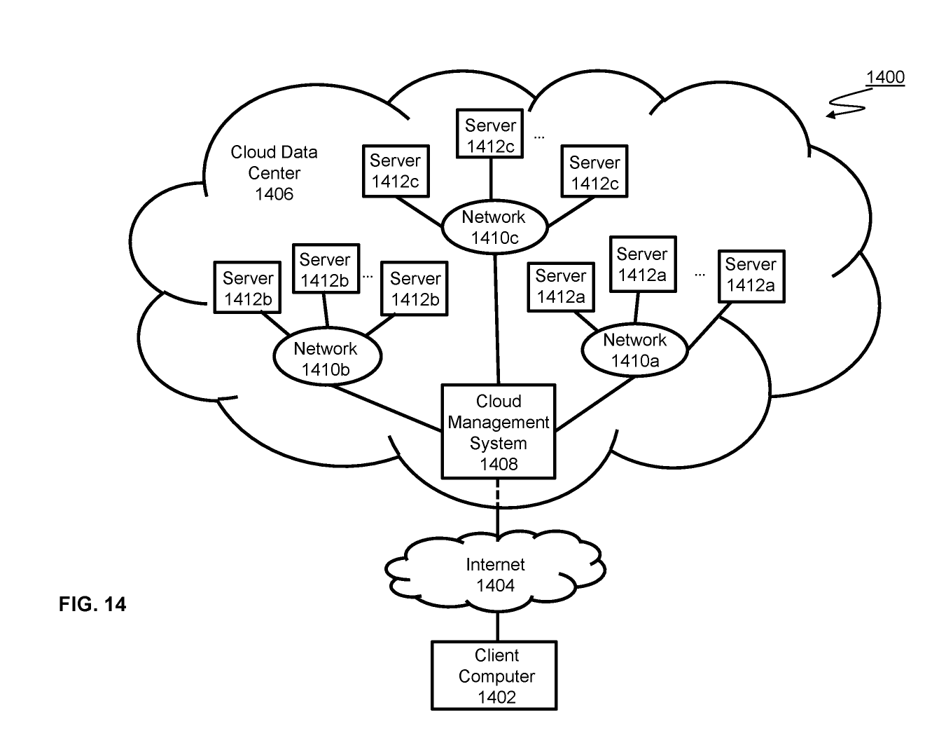

FIG. 14 illustrates an exemplary cloud computing system 1400 that may be used to implement all or a portion of the methods according to the present invention. The cloud computing system 1400 includes a plurality of interconnected computing environments. The cloud computing system 1400 utilizes the resources from various networks as a collective virtual computer, where the services and applications can run independently from a particular computer or server configuration making hardware less important.

Specifically, the cloud computing system 1400 includes at least one client computer 1402. The client computer 1402 may be any device through the use of which a distributed computing environment may be accessed to perform the methods disclosed herein, for example, the computing device described above in

FIG. 13, a traditional computer, portable computer, mobile phone, personal digital assistant, tablet to name a few. The client computer 1402 includes memory such as random access memory (“RAM”), read-only memory (“ROM”), mass storage device, or any combination thereof. The memory functions as a computer usable storage medium, otherwise referred to as a computer readable storage medium, to store and/or access computer software and/or instructions.

The client computer 1402 also includes a communications interface, for example, a modem, a network interface (such as an Ethernet card), a communications port, a PCMCIA slot and card, wired or wireless systems, etc. The communications interface allows communication through transferred signals between the client computer 1402 and external devices including networks such as the Internet 1404 and cloud data center 1406. Communication may be implemented using wireless or wired capability such as cable, fiber optics, a phone line, a cellular phone link, radio waves or other communication channels.

The client computer 1402 establishes communication with the Internet 1404—specifically to one or more servers—to, in turn, establish communication with one or more cloud data centers 1406. A cloud data center 1406 includes one or more networks 610a, 610b, 610c managed through a cloud management system 608. Each network 1410a, 1410b, 1410c includes resource servers 1412a, 1412b, 1412c, respectively. Servers 1412a, 1412b, 1412c permit access to a collection of computing resources and components that can be invoked to instantiate a virtual machine, process, or other resource for a limited or defined duration. For example, one group of resource servers can host and serve an operating system or components thereof to deliver and instantiate a virtual machine. Another group of resource servers can accept requests to host computing cycles or processor time, to supply a defined level of processing power for a virtual machine. A further group of resource servers can host and serve applications to load on an instantiation of a virtual machine, such as an email client, a browser application, a messaging application, or other applications or software.

The cloud management system 1408 can comprise a dedicated or centralized server and/or other software, hardware, and network tools to communicate with one or more networks 1410a, 1410b, 1410c, such as the Internet or other public or private network, with all sets of resource servers 1412a, 1412b, 1412c. The cloud management system 1408 may be configured to query and identify the computing resources and components managed by the set of resource servers 1412a, 1412b, 1412c needed and available for use in the cloud data center 1406. Specifically, the cloud management system 1408 may be configured to identify the hardware resources and components such as type and amount of processing power, type and amount of memory, type and amount of storage, type and amount of network bandwidth and the like, of the set of resource servers 1412a, 1412b, 1412c needed and available for use in the cloud data center 1406. Likewise, the cloud management system 1408 can be configured to identify the software resources and components, such as type of Operating System (“OS”), application programs, and the like, of the set of resource servers 1412a, 1412b, 1412c needed and available for use in the cloud data center 1406.

The present invention is also directed to computer products, otherwise referred to as computer program products, to provide software to the cloud computing system 1400. Computer products store software on any computer useable medium, known now or in the future. Such software, when executed, may implement the methods according to certain embodiments of the invention. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, optical storage devices, Micro-Electro-Mechanical Systems (“MEMS”), nanotechnological storage device, etc.), and communication mediums (e.g., wired and wireless communications networks, local area networks, wide area networks, intranets, etc.). It is to be appreciated that the embodiments described herein may be implemented using software, hardware, firmware, or combinations thereof.

The cloud computing system 1400 of FIG. 14 is provided only for purposes of illustration and does not limit the invention to this specific embodiment. It is appreciated that a person skilled in the relevant art knows how to program and implement the invention using any computer system or network architecture.

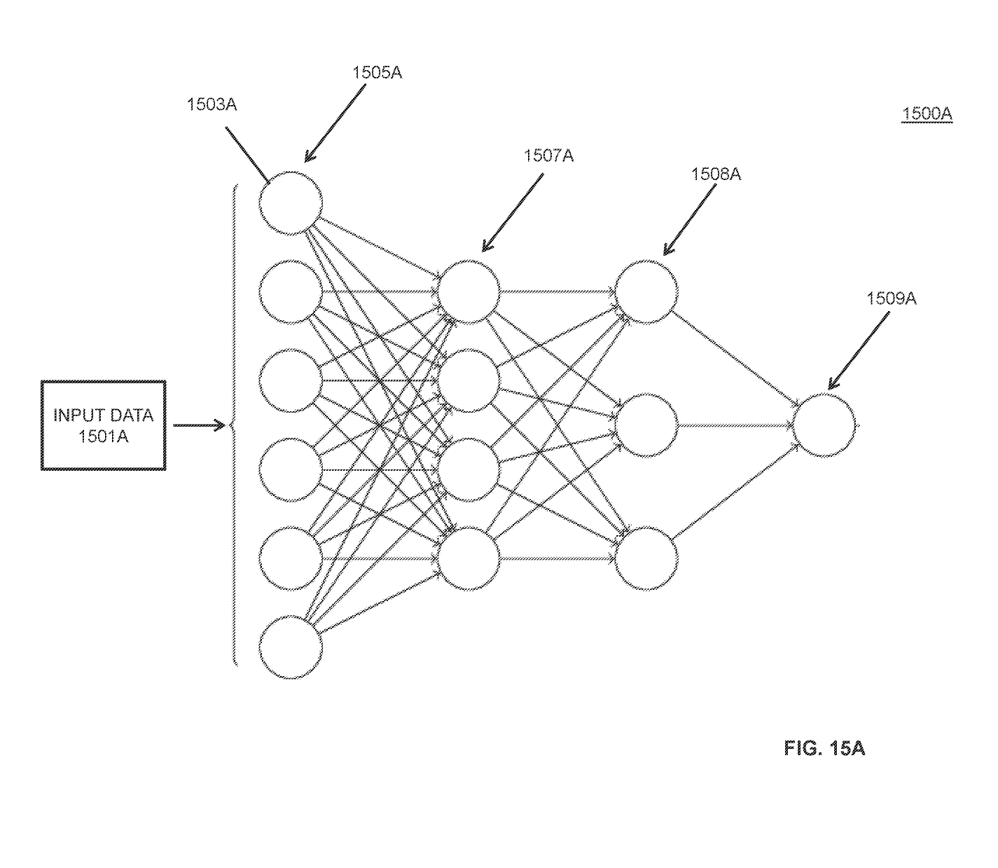

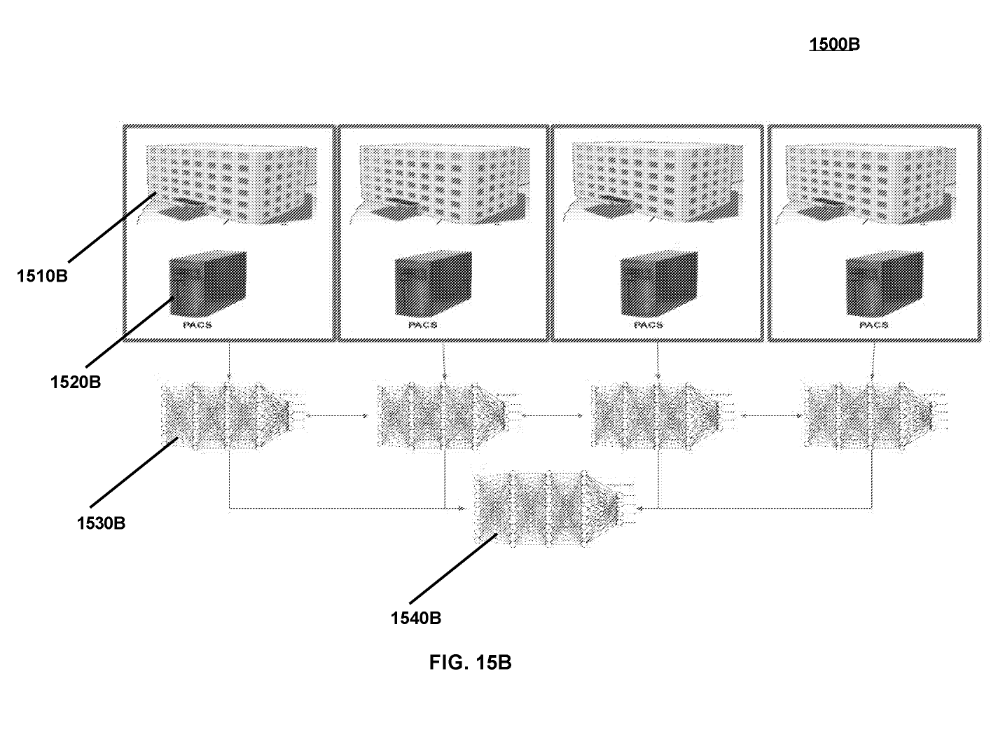

FIG. 15A illustrates an exemplary neural network 1500A that may be used to implement all or a portion of the methods according to the present invention. Specifically, the neural network 1500A can produce an output including to one or more structured data elements from a set of structured data elements that corresponds to an input—such as an image, an utterance, or a ROI.

In FIG. 15A, input data 1501A is first segmented into portions of data—for example pixel data—and inputted into a first layer 1503A—an input layer. Each layer in the neural network 1500A is made up of neurons 1505A that have learnable weights and biases. The middle layers—for example, 1507A and 1508A—are termed “hidden layers”. Each hidden layer is fully connected to all neurons in the first input layer 1503A. The neurons in each single layer of the hidden layers 1507A, 1508A function completely independently and do not share any connections. The last fully-connected layer 1509A is termed the “output layer” and may represent an identified structured data element. In certain embodiments, the neural network 1500A may be positioned between any two layers of a convolutional neural network such that the output layer 1509A acts as an input into another layer of a neural network.

In this embodiment, the hidden layers 1507A, 1508A neurons include a set of learnable filters which can process portions of the input data. As the input data is processed across each filter, dot products are computed between the entries of the filter and the input data 1501A to produce an activation map that gives the responses of that filter to the input data 1501A. The neural network 1500A will learn filters that activate when they detect a feature—such as an anatomical feature or pathologies displayed on an image.

The system can combine the activations maps produced in response to each filter at the output layer 1509A to produce a neural network model. For example, the output layer 1509A may identify a structured data element stored in a memory of the system. In certain embodiments, the system may automatically classify the medical condition and/or feature using a structured data element identified by the output layer 1509A. In other embodiments, the system may produce a report using the identified structured data element.

FIG. 15B illustrates one exemplary use of the cloud computing system 1400 of FIG. 14 for training and validating DL algorithms using medical images. In the DL cloud computing system (DL system) 1500B of FIG. 15B, each medical facility 1510B is equipped with DL technology that can train on the medical data local to that institution by processing the medical data through a DL algorithm. As shown in this embodiment, the medical data is stored in one or more Picture Archiving and Communication (PAC) servers 1520B that are accessible to the DL system 1500 B. This will create a collection of locally trained structured training data sets—DL models 1530B—, which may then be collected and enhanced for clinical use through a variety of approaches.

In certain embodiments, the DL system 1500 is a convolutional DL system, made up of neuron layers that may have learnable weights and biases for producing a convolution DL model. Each neuron in the network may receive some input and performs a dot product to produce the convolutional DL model. In some embodiments, the neurons at each layer consist of a set of learnable filters used to compute the dot product between the entries of the filter and the input data. As a result, the DL system 1500B learns filters that activate when it detects some specific type of feature at some spatial position in the input.

The DL system 1500B may then collect convolutional DL models from each medical facility 1510B and aggregate the weights from the convolutional filters features to form a single DL model 1540B from which the system 1500B can produce a federated DL algorithm.

One approach for training and validating the DL system 1500B includes averaging local trained data sets. Other approaches may include circulating the DL models 1530B among each medical facility 1510B, augmenting a DL model at a certain medical facility by processing new and/or additional data through a DL algorithm, and then either selecting the best DL model or using all DL models as training data for additional layers. The DL system 1500B may also implement an ensemble approach to aggregate the results of multiple DL models from each site. Common types of ensembles for machine learning include Bayes optimal classifier, Bootstrap aggregating, Boosting, Bayesian parameter averaging, Bayesian model combination, Bucket of models, and Stacking.

While the disclosure is susceptible to various modifications and alternative forms, specific exemplary embodiments thereof have been shown by way of example in the drawings and have herein been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

What is claimed is:

1. A method for producing at least one information component of a medical report, the method comprising:
accessing image data from a database, the image data including one or more medical images;
selecting a medical image;
outputting onto a display device at least one view of the selected medical image;
collecting gaze data of a user using a first input device;
receiving by a processor the gaze data and a second input from a second input device;
analyzing the gaze data and the second input, wherein the gaze data identifies a region of interest and the second input provides additional information corresponding to the region of interest;
combining the gaze data and the additional information to produce the at least one information component of the medical report;
assigning an identifier to the region of interest; and
communicating the at least one information component of the medical report to a user.

2. The method of claim 1, further comprising:
detecting patterns of visual fixations and saccades of the user.

3. The method of claim 1, further comprising:
locating clinical information corresponding to the region of interest, the clinical information including at least one of context information from studies of similar medical images, a patient's health, and a patient's medical history; and
wherein the at least one information component includes the clinical information.

4. The method of claim 1, further comprising:
locating a set of structured data elements in the database;
matching the region of interest to at least one term in the set of structured data elements; and
wherein the at least one information component includes the at least one term.

5. The method of claim 1, wherein the second input is an utterance.

6. The method of claim 5, further comprises:
locating a set of structured data elements in the database;
narrowing speech recognition software; and
matching at least one word of the utterance to a term in the set of structured data elements.

7. The method of claim 6, further comprising:
generating the at least one information component by populating a template with the at least one term.

8. The method of claim 1, wherein said assigning step includes associating a unique identifier specific to a patient.

9. The method of claim 1, further comprising:
locating a data set containing tagged images, wherein images in the data set include the identifier; and
processing the set of tagged image using a machine learning algorithm to produce a first machine learning model.

10. The method of claim 9, further comprising:
processing a plurality of machine learning models to produce a federated machine learning algorithm.

11. A system for producing at least one information component of a medical report, the system comprising:
a display device;
a first input device;
a second input device;
a non-transient, non-volatile memory;
a processor operatively coupled to the display device, the memory, the first input device, and the second input device, the memory including stored instructions that, when executed by the processor, cause the processor to:
access an image data set from a database in the memory, the image data set including a plurality of medical images;
select a medical image;
output onto the display device at least one view of a selected medical image;
collect gaze data of a user using the first input device;
receive the gaze data and a second input from the second input device;
analyze the gaze data and the second input, wherein the gaze data identifies a region of interest and the second input provides additional information corresponding to the region of interest;
combine the gaze data and the information from the second input to produce the at least one information component of the medical image;
assign an identifier to the region of interest; and
communicate the at least one information component of the medical report to a user.

12. The system of claim 11, wherein said first input device is a device for tracking movement of a user's eyes.

13. The system of claim 11, wherein said processor is further configured to:
locate clinical information corresponding to the region of interest, the clinical information including at least one of context information from studies of similar medical images, a patient's health, and a patient's medical history; and
wherein the at least one descriptive element includes the clinical information.

14. The system of claim 11, wherein said processor is further configured to:
locate a set of structured data elements in the database;
match the region of interest to at least one term in the set of structured data elements; and
wherein the at least one information component includes the at least one term.

15. The system of claim 11, wherein said second input device is a microphone.

16. The system of claim 15, wherein said processor is further configured to:
locate a set of structured data elements in the database;
narrow speech recognition software; and
match one word of the utterance to a term in the set of structured data elements.

17. The system of claim 16, wherein said processor is further configured to:
generate the at least one information component by populating at least one template with the at least one term.

18. The system of claim 11, further comprising a speaker, wherein the at least one information component is communicated audibly through the speaker.

19. The system of claim 11, wherein said processor is further configured to:
locate a data set containing tagged images, wherein at least one image in the data set includes the identifier; and
process the set of tagged images using a machine learning algorithm to produce a first machine learning model.

20. The system of claim 19, wherein said processor is further configured to:
process a plurality of machine learning models to produce a federated machine learning algorithm.
